(19) United States
(12) Patent Application Publication
(10) Pub. No.: US 2024/0153641 A1
(43) Pub. Date: May 9, 2024

(54) METHODS FOR GENOMIC IDENTIFICATION OF PHENOTYPE RISK

(71) Applicant: Progenic Genomics, Inc., Sausalito, CA (US)

(72) Inventor: Bradford A. Gulko, Sausalito, CA (US)

(21) Appl. No.: 18/204,127

(22) Filed: May 31, 2023

Related U.S. Application Data
(63) Continuation of application No. PCT/US21/61287, filed on Nov. 30, 2021.
(60) Provisional application No. 63/119,685, filed on Dec. 1, 2020.
(60) Provisional application No. 63/120,439, filed on Dec. 2, 2020.
(60) Provisional application No. 63/122,081, filed on Dec. 7, 2020.

(51) Int. Cl. G16H 50/30; C12Q 1/6806; C12Q 1/6869; G16B 20/20; G16B 30/20

(52) U.S. Cl. CPC G16H 50/30; C12Q 1/6806; C12Q 1/6869; G16B 20/20; G16B 30/20; C12Q 2600/156

(57) ABSTRACT

The present disclosure provides methods directed to the determination of a genomic sequence of an embryo. A method for determining a genomic sequence of an embryo may comprise isolating deoxyribonucleic acid (DNA) from the embryo, preparing a sequencing library, sequencing the sequencing library, and computer processing results of such sequencing (e.g., sequence reads) to determine the genomic sequence of the embryo.

### CROSS-REFERENCE

This application is a continuation of International Application No. PCT/US2021/061287, filed Nov. 30, 2021, which claims the benefit of U.S. Patent Application No. 63/119,685, filed Dec. 1, 2020, U.S. Patent Application No. 63/120,439, filed Dec. 2, 2020, and U.S. Patent Application No. 63/122,081, filed Dec. 7, 2020, each of which is incorporated by reference herein in its entirety.

### BACKGROUND

In vitro fertilization (IVF) may refer to a series of procedures used to help with fertility, prevent genetic problems, and assist with the conception of a child. Current embryonic genetic analysis may involve sequencing of a small amount of available genetic material in order to determine both euploidy (proper number of chromosomes) and the risk of a small number of identifiable genetic diseases. However, only a small number of cells may be available for study without harming the embryo. This small amount of genetic material may result in a large amount of noise during analysis. While the material may be chemically amplified to produce more DNA, current amplification processes may inject errors into the amplified product, which similarly impacts the accuracy of the final result.

### SUMMARY

The present disclosure provides methods for determining the genomic sequence of an embryo by simplifying comparison between genomes. The present disclosure provides methods for the aggregation and distillation of complex collections of genomic properties into a smaller set of phenotypical biases that may be used to select a genome from the collection of genomes for further operations. The present disclosure provides methods for the identification of genomic phenotype risk scores associated with an organism that possesses an expected genome. The present disclosure also provides methods leveraging replicon variation among a cohort to identify associations and risks for phenotypes, based on the genomics of an organism.

In some embodiments, the present disclosure provides a method for determining a genomic sequence of an embryo, comprising (a) isolating deoxyribonucleic acid (DNA) molecules from cells obtained or derived from a biopsy sample or culture sample of the embryo; (b) preparing a sequencing library from the DNA molecules or derivatives thereof; (c) sequencing the sequencing library to produce embryo-derived sequence reads; and (d) computer processing the embryo-derived sequence reads to determine the genomic sequence of the embryo using sequence information derived from one or more parents of the embryo. In some embodiments, the embryo is produced at least in part by in vitro fertilization of a sperm cell from a paternal subject and an egg cell from a maternal subject.

In some embodiments, the method further comprises sequencing second DNA molecules obtained or derived from the paternal subject or the maternal subject to produce parental-derived sequence reads, wherein the parental-derived sequence reads comprise paternal-derived sequence reads from the paternal subject or maternal-derived sequence reads from the maternal subject, respectively, and wherein (d) further comprises computer processing the embryo-derived sequence reads and the parental-derived sequence reads to determine the genomic sequence of the embryo.

In some embodiments, the parental-derived sequence reads comprise paternal-derived sequence reads from the paternal subject and maternal-derived sequence reads from the maternal subject. In some embodiments, the method further comprises performing contig assembly of individual sequence reads of the embryo-derived sequence reads and the parental-derived sequence reads to determine the genomic sequence of the embryo. In some embodiments, a portion of the genomic sequence of the embryo located between two breakpoints is determined based at least in part on a corresponding genomic sequence obtained from either the paternal-derived sequence reads or the maternal-derived sequence reads. In some embodiments, a plurality of portions of the genomic sequence of the embryo located between 3, 4, 5, 6, 7, 8, 9, 10, or more than 10 breakpoints are determined based at least in part on a corresponding genomic sequence obtained from either the paternal-derived sequence reads or the maternal-derived sequence reads. In some embodiments, a plurality of portions of the genomic sequence of the embryo located between 3, 4, 5, 6, 7, 8, 9, 10, or more than 10 breakpoints are determined based at least in part on a corresponding genomic sequence obtained from the paternal-derived sequence reads and the maternal-derived sequence reads.

In some embodiments, the embryo is a human embryo. In some embodiments, the embryo is a blastocyst. In some embodiments, the blastocyst is cultured for 1 day, 2 days, 3 days, 4 days, 5 days, 6 days, 7 days, 8 days, 9 days, or 10 days.

In some embodiments, the biopsy sample comprises trophectoderm cells of the blastocyst. In some embodiments, the culture sample comprises cells or cell-free DNA from culture media.

In some embodiments, the method further comprises computer processing at least a portion of the genomic sequence of the embryo to determine a presence or an absence of an aneuploidy or a genetic variation of the embryo. In some embodiments, the aneuploidy comprises trisomy 13, trisomy 18, trisomy 21, or a sex chromosome aneuploidy. In some embodiments, the genetic variation comprises a monogenic variant associated with a variant phenotype. In some embodiments, the variant phenotype comprises being affected by a disease or disorder or having an elevated risk of being affected by a disease or disorder.

In some embodiments, the method further comprises determining a number of alleles of the embryo comprising the monogenic variant. In some embodiments, the method further comprises determining whether the embryo is affected or at elevated risk of being affected by the variant phenotype, unaffected or at reduced risk of being affected by the variant phenotype, or a carrier of the variant phenotype, based at least in part on the determined number of alleles of the embryo comprising the monogenic variant. In some embodiments, the method further comprises computer processing the genomic sequence of the embryo to determine a risk distribution of each of a set of phenotypes.

In some embodiments, computer processing the genomic sequence of the embryo comprises using a trained machine learning algorithm. In some embodiments, the trained machine learning algorithm comprises a neural network, a support vector machine, a random forest, a generalized linear model, or a logistic regression.

In some embodiments, the risk distribution for a phenotype of the set of phenotypes is determined based at least in part on a combination of at least one of paternal haplo-blocks inherited by the embryo, maternal haplo-blocks inherited by the embryo, an observable paternal phenotype, and an observable maternal phenotype. In some embodiments, the risk distribution for a phenotype of the set of phenotypes is determined based at least in part on a combination of the paternal haplo-blocks inherited by the embryo, the maternal haplo-blocks inherited by the embryo, the observable paternal phenotype, and the observable maternal phenotype.

In some embodiments, the method further comprises computer processing the risk distributions of the set of phenotypes into a quantitative figure of merit indicative of an expected health of an offspring that develops from the embryo. In some embodiments, each of the risk distributions of the set of phenotypes contributes a positive expected value, a negative expected value, or a zero expected value toward the quantitative figure of merit. In some embodiments, at least one of the risk distributions of the set of phenotypes contributes a positive expected value toward the quantitative figure of merit. In some embodiments, the quantitative figure of merit comprises an expected number of quality adjusted life years of the offspring.

In some embodiments, the method further comprises determining a quantitative figure of merit for each of a plurality of embryos. In some embodiments, the quantitative figures of merit for the plurality of embryos are determined using a user-selected set of weights for each of at least one of the set of phenotypes.

In some embodiments, the method further comprises ordering or ranking individual embryos of the plurality of embryos based at least in part on the quantitative figures of merit for the individual embryos. In some embodiments, the method further comprises selecting an embryo from among the plurality of embryos based at least in part on the quantitative figures of merit for the individual embryos. In some embodiments, the selected embryo is implanted into a female subject, or wherein the selected embryo is vitrified, incubated, cultivated, stored, investigated, manipulated, treated or discarded. In some embodiments, the method further comprises implanting the selected embryo into the female subject.

In some embodiments, the sequencing library in (b) is prepared without use of nucleic acid amplification. In some embodiments, the genomic sequence of the embryo is determined at an accuracy of at least about 99%, about 99.9%, about 99.99%, about 99.999%, about 99.9999%, about 99.99999%, or about 99.999999%. In some embodiments, the genomic sequence of the embryo is at least 90%, at least 95%, at least 99%, or at least 99.9% of a whole genomic sequence of the embryo. In some embodiments, the genomic sequence of the embryo is a whole genomic sequence or a substantially whole genomic sequence of the embryo.

In some embodiments, the present disclosure provides a computer-implemented method for determining a genomic sequence of an embryo, comprising: (a) receiving, by a computer, embryo-derived sequence reads of an embryo, wherein the embryo-derived sequence reads are generated by sequencing deoxyribonucleic acid (DNA) molecules that are isolated or derived from cells obtained or derived from a biopsy sample or a culture sample of the embryo; (b) receiving, by the computer, sequence information derived from one or more parents of the embryo; and (c) computer processing the embryo-derived sequence reads to determine the genomic sequence of the embryo using the sequence information derived from the one or more parents of the embryo. In some embodiments, the embryo is produced at least in part by in vitro fertilization of a sperm cell from a paternal subject and an egg cell from a maternal subject.

In some embodiments, the method further comprises receiving parental-derived sequence reads comprising paternal-derived sequence reads from the paternal subject or maternal-derived sequence reads from the maternal subject, respectively, and wherein (c) further comprises computer processing the embryo-derived sequence reads and the parental-derived sequence reads to determine the genomic sequence of the embryo. In some embodiments, the parental-derived sequence reads comprise paternal-derived sequence reads from the paternal subject and maternal-derived sequence reads from the maternal subject.

In some embodiments, the method further comprises performing contig assembly of individual sequence reads of the embryo-derived sequence reads and the parental-derived sequence reads to determine the genomic sequence of the embryo. In some embodiments, a portion of the genomic sequence of the embryo located between two breakpoints is determined based at least in part on a corresponding genomic sequence obtained from either the paternal-derived sequence reads or the maternal-derived sequence reads. In some embodiments, a plurality of portions of the genomic sequence of the embryo located between 3, 4, 5, 6, 7, 8, 9, 10, or more than 10 breakpoints are determined based at least in part on a corresponding genomic sequence obtained from either the paternal-derived sequence reads or the maternal-derived sequence reads. In some embodiments, a plurality of portions of the genomic sequence of the embryo located between 3, 4, 5, 6, 7, 8, 9, 10, or more than 10 breakpoints are determined based at least in part on a corresponding genomic sequence obtained from the paternal-derived sequence reads and the maternal-derived sequence reads.

In some embodiments, the embryo is a human embryo. In some embodiments, the embryo is a blastocyst. In some embodiments, the blastocyst is cultured for 1 day, 2 days, 3 days, 4 days, 5 days, 6 days, 7 days, 8 days, 9 days, or 10 days. In some embodiments, the biopsy sample comprises trophectoderm cells of the blastocyst. In some embodiments, the culture sample comprises cells or cell-free DNA from culture media.

In some embodiments, the method further comprises computer processing at least a portion of the genomic sequence of the embryo to determine a presence or an absence of an aneuploidy or a genetic variation of the embryo. In some embodiments, the aneuploidy comprises trisomy 13, trisomy 18, trisomy 21, or a sex chromosome aneuploidy. In some embodiments, the genetic variation comprises a monogenic variant associated with a variant phenotype. In some embodiments, the variant phenotype comprises being affected by a disease or disorder or having an elevated risk of being affected by a disease or disorder.

In some embodiments, the method further comprises determining a number of alleles of the embryo comprising the monogenic variant. In some embodiments, the method further comprises determining whether the embryo is affected or at elevated risk of being affected by the variant phenotype, unaffected or at reduced risk of being affected by the variant phenotype, or a carrier of the variant phenotype, based at least in part on the determined number of alleles of the embryo comprising the monogenic variant.

In some embodiments, the method further comprises computer processing the genomic sequence of the embryo to determine a risk distribution of each of a set of phenotypes. In some embodiments, computer processing the genomic sequence of the embryo comprises using a trained machine learning algorithm. In some embodiments, the trained machine learning algorithm comprises a neural network, a support vector machine, a random forest, a generalized linear model, or a logistic regression.

In some embodiments, the risk distribution for a phenotype of the set of phenotypes is determined based at least in part on a combination of at least one of paternal haplo-blocks inherited by the embryo, maternal haplo-blocks inherited by the embryo, an observable paternal phenotype, and an observable maternal phenotype. In some embodiments, the risk distribution for a phenotype of the set of phenotypes is determined based at least in part on a combination of the paternal haplo-blocks inherited by the embryo, the maternal haplo-blocks inherited by the embryo, the observable paternal phenotype, and the observable maternal phenotype. In some embodiments, the method further comprises computer processing the risk distributions of the set of phenotypes into a quantitative figure of merit indicative of an expected health of an offspring that develops from the embryo. In some embodiments, each of the risk distributions of the set of phenotypes contributes a positive expected value, a negative expected value, or a zero expected value toward the quantitative figure of merit. In some embodiments, at least one of the risk distributions of the set of phenotypes contributes a positive expected value toward the quantitative figure of merit.

In some embodiments, the quantitative figure of merit comprises an expected number of quality adjusted life years of the offspring. In some embodiments, the method further comprises determining a quantitative figure of merit for each of a plurality of embryos. In some embodiments, the quantitative figures of merit for the plurality of embryos are determined using a user-selected set of weights for each of at least one of the set of phenotypes.

In some embodiments, the method further comprises ordering or ranking individual embryos of the plurality of embryos based at least in part on the quantitative figures of merit for the individual embryos. In some embodiments, the method further comprises selecting an embryo from among the plurality of embryos based at least in part on the quantitative figures of merit for the individual embryos.

In some embodiments, the selected embryo is implanted into a female subject, or wherein the selected embryo is vitrified, incubated, cultivated, stored, investigated, manipulated, treated or discarded. In some embodiments, the method further comprises implanting the selected embryo into the female subject.

In some embodiments, the embryo-derived sequence reads are generated without use of nucleic acid amplification. In some embodiments, the genomic sequence of the embryo is determined at an accuracy of at least about 99%, about 99.9%, about 99.99%, about 99.999%, about 99.9999%, about 99.99999%, or about 99.999999%. In some embodiments, the genomic sequence of the embryo is at least 90%, at least 95%, at least 99%, or at least 99.9% of a whole genomic sequence of the embryo. In some embodiments, the genomic sequence of the embryo is a whole genomic sequence or a substantially whole genomic sequence of the embryo.

In some embodiments, the present disclosure provides a method for providing a selection of an embryo from a set of sibling embryos, comprising: (a) obtaining a first sequence data set generated upon sequencing one or more nucleic acid molecules obtained from the embryo, which first sequence data set is not a whole genome of said embryo; (b) computer processing the first sequence data set with sequence information obtained from one or more parents of the sibling embryos to yield a second sequence data set, which second sequence data set spans a greater genomic window than the first sequence data set; and (c) computer processing the second sequence data set or derivative thereof to provide the selection of said embryo from the set of sibling embryos. In some embodiments, the set of sibling embryos is produced at least in part by in vitro fertilization of a set of sperm cells from a paternal subject and a set of egg cells from a maternal subject.

In some embodiments, the method further comprises receiving parental-derived sequence reads comprising paternal-derived sequence reads from the paternal subject or maternal-derived sequence reads from the maternal subject, respectively, and wherein (c) further comprises computer processing the parental-derived sequence reads to provide the selection of said embryo from the set of sibling embryos. In some embodiments, the parental-derived sequence reads comprise paternal-derived sequence reads from the paternal subject and maternal-derived sequence reads from the maternal subject.

In some embodiments, the method further comprises determining a genomic sequence of the embryo, and providing the selection of said embryo from the set of sibling embryos based at least in part on the determined genomic sequence of the embryo. In some embodiments, the method further comprises performing contig assembly of individual sequence reads of the embryo-derived sequence reads and the parental-derived sequence reads to determine the genomic sequence of the embryo.

In some embodiments, a portion of the genomic sequence of the embryo located between two breakpoints is determined based at least in part on a corresponding genomic sequence obtained from either the paternal-derived sequence reads or the maternal-derived sequence reads. In some embodiments, a plurality of portions of the genomic sequence of the embryo located between 3, 4, 5, 6, 7, 8, 9, 10, or more than 10 breakpoints are determined based at least in part on a corresponding genomic sequence obtained from either the paternal-derived sequence reads or the maternal-derived sequence reads. In some embodiments, a plurality of portions of the genomic sequence of the embryo located between 3, 4, 5, 6, 7, 8, 9, 10, or more than 10 breakpoints are determined based at least in part on a corresponding genomic sequence obtained from the paternal-derived sequence reads and the maternal-derived sequence reads.

In some embodiments, the embryo is a human embryo. In some embodiments, the embryo is a blastocyst. In some embodiments, the blastocyst is cultured for 1 day, 2 days, 3 days, 4 days, 5 days, 6 days, 7 days, 8 days, 9 days, or 10 days. In some embodiments, the biopsy sample comprises trophectoderm cells of the blastocyst. In some embodiments, the culture sample comprises cells or cell-free DNA from culture media.

In some embodiments, the method further comprises computer processing at least a portion of the genomic sequence of the embryo to determine a presence or an absence of an aneuploidy or a genetic variation of the embryo. In some embodiments, the aneuploidy comprises trisomy 13, trisomy 18, trisomy 21, or a sex chromosome aneuploidy. In some embodiments, the genetic variation comprises a monogenic variant associated with a variant phenotype. In some embodiments, the variant phenotype comprises being affected by a disease or disorder or having an elevated risk of being affected by a disease or disorder.

In some embodiments, the method further comprises determining a number of alleles of the embryo comprising the monogenic variant. In some embodiments, the method further comprises determining whether the embryo is affected or at elevated risk of being affected by the variant phenotype, unaffected or at reduced risk of being affected by the variant phenotype, or a carrier of the variant phenotype, based at least in part on the determined number of alleles of the embryo comprising the monogenic variant.

In some embodiments, the method further comprises computer processing the genomic sequence of the embryo to determine a risk distribution of each of a set of phenotypes. In some embodiments, computer processing the genomic sequence of the embryo comprises using a trained machine learning algorithm. In some embodiments, the trained machine learning algorithm comprises a neural network, a support vector machine, a random forest, a generalized linear model, or a logistic regression.

In some embodiments, the risk distribution for a phenotype of the set of phenotypes is determined based at least in part on a combination of at least one of paternal haplo-blocks inherited by the embryo, maternal haplo-blocks inherited by the embryo, an observable paternal phenotype, and an observable maternal phenotype. In some embodiments, the risk distribution for a phenotype of the set of phenotypes is determined based at least in part on a combination of the paternal haplo-blocks inherited by the embryo, the maternal haplo-blocks inherited by the embryo, the observable paternal phenotype, and the observable maternal phenotype. In some embodiments, the method further comprises computer processing the risk distributions of the set of phenotypes into a quantitative figure of merit indicative of an expected health of an offspring that develops from the embryo.

In some embodiments, each of the risk distributions of the set of phenotypes contributes a positive expected value, a negative expected value, or a zero expected value toward the quantitative figure of merit. In some embodiments, at least one of the risk distributions of the set of phenotypes contributes a positive expected value toward the quantitative figure of merit. In some embodiments, the quantitative figure of merit comprises an expected number of quality adjusted life years of the offspring.

In some embodiments, the method further comprises further comprising determining a quantitative figure of merit for each of a plurality of embryos. In some embodiments, the quantitative figures of merit for the plurality of embryos are determined using a user-selected set of weights for each of at least one of the set of phenotypes.

In some embodiments, the method further comprises ordering or ranking individual embryos of the plurality of embryos based at least in part on the quantitative figures of merit for the individual embryos. In some embodiments, the method further comprises selecting an embryo from among the plurality of embryos based at least in part on the quantitative figures of merit for the individual embryos. In some embodiments, the selected embryo is implanted into a female subject, or wherein the selected embryo is vitrified, incubated, cultivated, stored, investigated, manipulated, treated or discarded. In some embodiments, the method further comprises implanting the selected embryo into the female subject.

In some embodiments, the sequencing library in (b) is prepared without use of nucleic acid amplification. In some embodiments, the second sequence data set is determined at an accuracy of at least about 99%, about 99.9%, about 99.99%, about 99.999%, about 99.9999%, about 99.99999%, or about 99.999999%. In some embodiments, the second sequence data set is at least 90%, at least 95%, at least 99%, or at least 99.9% of a whole genomic sequence of the embryo.

In some embodiments, the present disclosure provides for a non-transitory computer-readable medium comprising machine-executable code that, upon execution by one or more computer processors, implements a method for determining a genomic sequence of an embryo, the method comprising: (a) receiving embryo-derived sequence reads of an embryo, wherein the embryo-derived sequence reads are generated by sequencing deoxyribonucleic acid (DNA) molecules that are isolated or derived from cells obtained or derived from a biopsy sample or a culture sample of the embryo; (b) receiving sequence information derived from one or more parents of the embryo; and (c) processing the embryo-derived sequence reads to determine the genomic sequence of the embryo using the sequence information derived from the one or more parents of the embryo.

In some embodiments, the present disclosure provides a method for a non-transitory computer-readable medium comprising machine-executable code that, upon execution by one or more computer processors, implements a method for providing a selection of an embryo from a set of sibling embryos, the method comprising: (a) obtaining a first sequence data set generated upon sequencing one or more nucleic acid molecules obtained from the embryo, which first sequence data set is not a whole genome of said embryo; (b) processing the first sequence data set with sequence information obtained from one or more parents of the sibling embryos to yield a second sequence data set, which second sequence data set spans a greater genomic window than the first sequence data set; and (c) processing the second sequence data set or derivative thereof to provide the selection of said embryo from the set of sibling embryos.

In some embodiments, the present disclosure provides a method for providing a selection of an embryo from a set of sibling embryos, comprising analyzing embryos from the set of embryos to (i) calculate a quality adjusted life expectancy of the embryos, and (ii) provide the selection of the embryo from the set of embryos, which embryo has a highest quality adjusted life expectancy among other embryos of the set of embryos as determined at an accuracy greater than about 80%. In some embodiments, embryos are selected based at least in part on a combination of at least one of paternal haplo-blocks inherited by the embryo, maternal haplo-blocks inherited by the embryo, an observable paternal phenotype, and an observable maternal phenotype. In some embodiments, embryos are selected based at least in part on a combination of the paternal haplo-blocks inherited by the embryo, the maternal haplo-blocks inherited by the embryo, the observable paternal phenotype, and the observable maternal phenotype.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A illustrates sample mappings from genomes represented as reference sequence segments, to genomic properties.

FIG. 1B illustrates genome segmentation by replicon.

FIG. 2 provides a flowchart illustrating one example of a method by which a model and associated values may be generated.

FIG. 3 provides a flowchart illustrating one example of a method by which models may be applied to generate simplified descriptions of genomes.

FIG. 4A represents the statistical relationship between organism genomes and phenotype risk scores.

FIG. 4B represents the statistical relationship between organism genomes that underlies methods and systems of the present disclosure.

FIG. 4C describes a method used to identify phenotype risk scores from organism genomes.

FIG. 5 provides a flowchart illustrating one example of a method by which models may be developed that may be used in methods and systems of the present disclosure.

FIG. 6 provides a flowchart illustrating one example of a method by which models may be applied using methods and systems of the present disclosure to generate improved phenotypic risk scores.

FIG. 7 illustrates a method for generating and applying small variant analysis.

FIG. 8 shows an example of the application of replicons to develop association studies and risk estimates.

FIG. 9 shows a computer system that is programmed or otherwise configured to implement methods provided herein.

FIG. 10 illustrates a method for identifying embryonic genomic sequences, determining risk distributions from the genomic sequences and other information, and aggregating risk distributions into a report for use in IVF.

In these drawings, dashed lines represent elements that may be present in some described embodiments but absent in others. Diagonal hashing represents a latent (unobserved) set of variables in a statistical model while a clear background represents an observed set of variables. A dotted outline represents a process which may be repeated in the course of developing an output.

### DETAILED DESCRIPTION

Nearly every organism has a genetic code that is shared by all of the cells in its body. This code may be identified using various approaches with single-molecule precision. While this code may be a primary determinant of species separation and heritable features within a species, the mapping from genome to identifiable phenotypic features may be poorly understood.

Genomes of individuals within an animal species may be similar, allowing for the creation of a reference genome for each species that allows the characterization of an individual in terms of deviations from that reference. For example, hg18, hg19, and GRCh38, are three progressively refined versions of the human genomic reference. Each such reference may define a coordinate system, which allows for the identification of genomic properties associated with positions in that coordinate system.

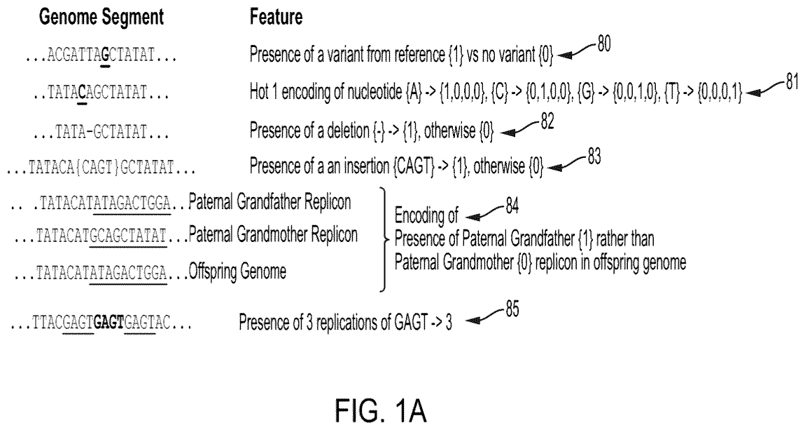

Observable traits in an organism, called phenotypes, can be transmitted from parents to offspring. A central mechanism for inheritance of such traits may be the collection of genomic material that is transferred from parents to offspring during procreation. In mammalian genomes, organisms generally contain two copies of each chromosome, one derived from each parent. For procreation, one copy of each chromosome is provided from each parent to the child. However, the specific chromosome provided may be a mixture of the genetic material of both chromosomes possessed by that parent, via a process called crossover, sometimes referred to as recombination. As a result, each embryo may inherit a single chromosome from each parent, but the chromosome inherited from a parent is potentially a mosaic composed of the genetic material inherited from that parent's own parents (the embryo's grandparents) (FIG. 1A-B).

Humans are a biallelic species with normal cells having 22 pairs of autosomal chromosomes and one pair of sex chromosomes. FIG. 1B, 83 shows labeling of each chromosome identifier in a central circle roughly corresponding to a centromere. Each chromosome is comprised of a pair of chromatid arms, and may be homologous in the cases of the autosome and the female XX chromosomes. One copy of each chromatid element may be inherited from each parent, and matching elements from each parent may fuse to make a chromosome comprised of one chromatid from each parent. However, the specific chromatid inherited from a parent is a mosaic of their own chromosome pair (FIG. 1B). In FIG. 1B, 80 the paternal element is a single chromatid, composed of a mosaic of the two paternal chromatids (FIG. 1B, 81). In some cases, a chromatid element is passed directly from a whole parental chromatid (e.g. FIG. 1B, 84 or the Y sex chromosome). Crossover allows for these mosaic patterns, and typically between 0 and 10 such crossovers may occur in each chromatid arm, meaning that a single chromatid inherited from a single parent, is actually a mosaic composed of the chromatid pair inherited by that parent from their own parents. Each contiguous chromatid section inherited from a single parent is referred to herein as a replicon. This crossover serves as a major source of diversity within a species and occurs in a wide variety of animal and plant species. In humans, there may be between 15 and 150 such crossovers per offspring. This mosaic, combined with the selection of one chromatid from each parent, seems to provide the primary source of genomic variation in traits among offspring from a single pair of parents.

The combinations of chromosomes, from parents, mosaics from grandparents, and variation inherited from neither parent (e.g., a de-novo mutation) gives rise to genetic diversity among full siblings. However, the restriction on variation as primarily sourcing from the parents limited genomic material provides similarity shared by related family members.

The genomic sequence of an embryo composed of fewer than a hundred cells may be estimated by combining a small amount of embryonic genetic material with a larger amount of parental material. However, the way in which the particular combinations of parental genomic material manifest as phenotypes may not be clear for many phenotypes. Some genomic variations may cause specific variant phenotypes or diseases in humans such as Huntington's disease, Huntington's chorea, and Marfan Syndrome (autosomal dominant diseases which requires only one copy of the pathogenic allele); cystic fibrosis and Tay-Sachs disease (autosomal recessive diseases which require two copies of the pathogenic allele); or Down Syndrome and Edwards Syndrome (diseases caused by aneuploidy, having missing or extra chromosomes). Some aneuploidies may be trisomies, wherein there are three copies of a gene (e.g. trisomy 13, trisomy 18, trisomy 21, or a sex chromosome trisomy). Some aneuploidies may be monosomies, wherein there is one copy of a gene (e.g., a sex chromosome monosomy). Some genomic variations may cause monogenic phenotypes, i.e., phenotypes determined by the alleles of one gene. Alternatively, some genomic variations may cause polygenic phenotypes, i.e., phenotypes determined by the alleles of multiple genes.

While phenotypes include the manifestation of or predisposition for diseases, they also include non-pathological traits such as weight, height, facial shape, and skin tone. Some genomic analysis techniques (e.g., polygenic risk score (PRS), genome-wide association studies (GWAS), etc.) may associate individual genomic properties or patterns of genomic properties with observable phenotypes and are also used as methods for associating genomic features with a propensity towards individual phenotypes.

When direct causal relations between genes and phenotypes are not known, association studies may be performed to relate associated patterns of genomic properties to phenotypes. For example, to obtain a PRS, a linear map can be created by taking known variants of a human reference genome (e.g., hg19), converting them to binary values, placing statistical weight on the presence of each particular variant, and determining a numerical score for each phenotypic variation or set of phenotypic variations. Creating such a linear map simplifies genetic analysis by associating the set of phenotypic variations with a numerical score, where that score represents the risk of obtaining the phenotype, given the set of variations. The scoring may be calibrated in such a way that the score has a value between 0.0 and 1.0 (e.g. by applying a Logistic link function in a generalized linear model), and which serves as an estimate of the risk of observing a phenotype conditioned on observing the set of variations.

Phenotype prediction and association studies may focus primarily on single nucleotide variations and small structural arrangements in GWAS and PRS studies, rather than replicon inheritance. While some elements of lineage aware PRS/GWAS analyses have been documented, they may be focused on reducing spurious correlations within populations rather than the essential mechanism of replicon recombination. Such recombination provides powerful genomic variation that drives phenotype variation, particularly with related families. Over the number of all human replicons, estimates of large-scale replicon segments range from thousands to tens of thousands, far lower than the tens of millions of small variations which occur. The combination of greater generational variation coupled with lower numbers of potentially confounding cofactors promises statistical models of far greater predictive power and predictive utility.

Using PRS and GWAS, many scores and association maps may be separately created for various phenotypes of interest. When a single genome is analyzed, many PRS analyses may be generated, each representing a risk associated with a particular phenotype and/or condition. However, each PRS may represent a different assessment, and provide a different level of confidence. Furthermore, the phenotypes themselves may have varying levels of relevance depending of the circumstances for using a PRS. Non-limiting examples of situations wherein decisions are made between different sets of PRS analyses include when a prospective breeder is seeking to create more healthy livestock; when a genomic edit is being considered, and a most favorable outcome phenotype is desired; or when a prospective human parent is selecting from among embryos generated during IVF.

While genomic features may represent a propensity or bias in the development of a phenotype (e.g., the presence of a phenotype or the magnitude of a phenotypes), development and environmental interaction also affect phenotype emergence.

Both PRS and GWAS techniques aggregate across environmental conditions and ignore specific details of subjects' environment which can manifest biologically via epigenetics. PRS and GWAS techniques may be applied to a variety of demographics and species, yet they are poor individual predictors for complex traits such as human adult height or weight. Presence or absence of a disease may be endemic to a genomic condition (e.g. Huntington's Disease, with high penetrance), or alternatively may be triggered by an environmental condition, also influenced by innate genomic susceptibility, as in the case of lactose-based gastronomic distress.

As related offspring are often exposed to similar environmental conditions as the parents, the presence of a close familial phenotype (e.g. a parental phenotype) may be informative on a broad range of environmental and developmental factors. Parental phenotype may, therefore, may serve as, e.g., a proxy, to estimate actual risk of a descendent phenotype.

Assisted reproductive technologies allow for the identification of an embryonic genome prior to the implantation, development, and rearing of an organism to maturity. Alternatively, embryonic genomes can be identified for purposes of vitrification, i.e., the process whereby embryos or eggs are frozen and stored for later use. Alternatively, the embryonic genomes can be identified for purposes of incubation, cultivation, storage, investigation, manipulation, treatment, or disposal. The ability to interpret an embryonic genome may allow selection based on expected traits such as disease resistance. A clearer understanding of the relationship between the sequenced genome and phenotypic traits may be of tremendous value in a diverse range of fields including the ability to cultivate desirable traits in livestock without the cost and delay of having to raise livestock to maturity; the assessment of candidate embryonic genomes; the assessment of disease-risk phenotypes in humans; the prediction of where make edits in human cells in order to treat or correct genetic diseases; or the assessment of human characteristics during assisted reproduction to avoid diseases in offspring and favor healthy traits.

A central method in understanding the mapping between traits and genotypes involves identifying specific phenotypic variation associated with specific genomic variation. However, species genomes have limited variation. For example, less than 10% of the human genome (estimates may range from 2-8%) is expected to be under selective pressure associated with genomic function. Novel genomic variation is introduced slowly into a species, with only 20-100 variants (in a genome of approximately 3 billion positions) introduced in each human generation that are not attributable to either parent. Many variations in biologically active regions are never observed because they introduce lethal changes, and embryos inheriting them never mature to the point where the novel variations are measured. Considering 50 variants per generation, with only 10% in functional areas, that leaves only 5 functional variants introduced per generation, assuming a random distribution of variation. However, variation is not observed uniformly across the genome, and the number of impactful variants per generation in functional areas may be substantially lower. Over a course of millions of years, and many thousands of generations, such random mutation introduces variation that can be associated with survivable phenotypic properties. The absence of variation that might give rise to lethal changes becomes more apparent with time and diversity in population and has been a popular topic of contemporary literature. In humans, single nucleotide polymorphism databases (e.g., dbSNP) provide approximately 40.6 million sites in the autosomal genome that are subject to “common” variation, and which represent about 1.4% of all genomic positions. Common variation is variation that is expected to occur in more than 1% of the population. When also considering rare variants, this number can grow by a factor of more than 10, up to and including more than 20% of all genomic positions. With approximately 2.8 billion autosomal genomic positions identified in human reference genomes (e.g., hg19), this leads to a bewildering and statistically challenging problem of predictively mapping patterns of variations to observed phenotypes. However, there is a second source of variation and constraint that can operate in multi-allelic species such as humans. This form of variation is called is meiotic recombination. Meiotic recombination occurs at reproduction, and is highly constrained along the genome, while at the same time being more common than random mutation in each reproductive generation.

In an assisted-fertility vetting, it may be desirable to understand the predisposition towards phenotypes so embryos can be ranked according to risks of desired and undesired phenotypes, such as predisposition towards disease (or resistance to diseases). Such genomic analysis of phenotypes has a variety of uses. In a medical or scientific context, the ability to prioritize genetically altered cells for treatment, investigation, or scientific inquiry may yield tremendous increases in safety and efficiency. In a livestock setting this might be used to more efficiently breed healthier and larger cattle or faster racehorses. In a human assisted reproduction (particularly In-Vitro Fertilization or IVF) it might be used to help reduce indications of diseases or select predisposition towards relevant traits for family balancing.

At present, human-assisted reproduction genomic tests for embryos generated during assisted reproduction in humans may favor tests for embryonic viability, rather than eventual adult health. However, genomic medicine has developed a range of models, each accepting a collection of genomic properties and identifying a risk of a specific phenotype or small collection of phenotypes. Many such tests are statically significant but have low individual predictive power. If these tests are collectively applied to the genome inferred from an embryo, a collection of risk scores may be generated. However, this collection of scores (potentially hundreds, thousands, or more) leaves a prospective parent with a large array of biases and traits to consider, without a method of aggregating these scores into a small number of classes that a parent might select away from, such as predisposition towards worse mental health or a predisposition towards lowered physical health.

In some embodiments, reduction of collections of phenotypic risk scores to simpler traits can be considered as a reduction from a large collections of scores to a small collection of properties. For example, the Meyer-Briggs type indicator (MBTI) test summarizes personalities according to a set of four types, each type having a score from a low value such as 0 (representing one pole of a trait such as introversion) to a high vale representing the opposing pole of the trait (such as extraversion).

In some embodiments, reduction of collections allows mapping of PRS to a single score which can be an estimate of some desired figure of merit, such as expected lifespan or medical quality of life, allowing the ordered ranking of each genome according to that score. In the case that PRS represent risks of disease an aggregate being assessed for IVF, the figure of merit may represent overall expected resistance or susceptibility to disease, and the best score may be taken as most likely to be healthy and selected for implantation.

In some embodiments, a more definitive measure may aggregate all of the various risks score into a single measure (or figure of merit) as an estimate of embryonic quality that can rank available embryos for implantation. While this embodiment focuses on human assisted reproduction, this technology can also be used to improve efficiency in breeding stock or sport animals by selecting and developing the most promising embryos. Furthermore, this technology can be used in the development of desirable characteristics in cell lines by assessing genomes resulting from genomic edits, assessing likely edit sites, or selecting cell culture that are most likely to have desirable profiles of phenotype qualities including immune response, antigen compatibility, or native disease resistance.

What is needed to improve the individual predictive power of phenotypic risk scores (e.g., polygenic risk scores), is a way of integrating both the genomic propensity for a phenotype along with the environmental factors that may impact the manifestation of that phenotype.

The present disclosure provides a method and system for simplifying the comparison between genomes, which may comprise a defined collection of genomic properties with each collection representing a single genome, a number of risk score models for phenotypes. In some embodiments, each risk score model maps the collection of genomic properties to a weight distribution model representing a projected phenotype distribution, and a dimension reduction model for mapping said collection of phenotype distributions to a simpler collection of trait distributions. In the case that the simpler collection of trait distributions is univariate, a centrality parameter may be generated for the distribution and associated with the corresponding genome. By assessing each of a collection of genomes according to this measure, comparisons among genomes may be simplified to comparisons of simpler traits. In the case that each trait distribution may be reduced single centrality parameter (as a figure of merit) the genomes may be ranked in order of decreasing merit with the most meritorious selected for further use. In some embodiments, each genome may belong to an embryo, genomic properties are variations from a reference, and risk scores represent standard PRS for diseases associated with each collection of variants. In this case, the figure of merit may be considered a medical quality of life model and a single value for expected quality of life derived for each genome by summing the individual expected contribution to quality of life from each disease across the polygenic risk of that disease.

The present disclosure also provides methods and systems that address inclusion of environmental information such as parental phenotypes along with embryonic genotype in the risk assessment for eventual development of a phenotype. In some embodiments, methods and systems of the present disclosure may be applied to embryo analysis for assisted reproduction in humans via IVF. In particular, environmental exposures of related organisms may be incorporated along with the genome of an organism to improve the adaptability of phenotype risk scores over those generated solely from an organism's genome. In some embodiments, this allows for improved identification of phenotypic risks for genomes associated with embryos by incorporating phenotype information of parents. In some embodiments, the genotype risk scores may be used for human assisted reproduction, by improving the predictive power of phenotype risk scores, allowing prospective parents more information in the selection of which embryos to implant.

The present disclosure provides methods for incorporating environmental information into the process of assessing the likelihood of phenotypes manifesting from an organismal genome. Environmental information is incorporated along with genomic properties of a related set of genomes and a target genome into a statistical model, said model accepting a target genome, related genomes, and a collection of related environmental values to produce a collection of risk scores representing a distribution over identified target phenotypes. In some embodiments, a joint distribution is produced allowing the calculation of a weight distribution for each target phenotype in each environmental condition. In other embodiments, phenotypes of related genomes serve as an informative proxy for external environmental factors that have not been explicitly identified. In some embodiments, this analysis might be applied to assisted reproductive technologies to combine parental phenotype information with embryonic genome properties to estimate risk of phenotype development as the embryo develops.

The methods and systems of the present disclosure may incorporate small variations into replicon-based variational analysis. In some embodiments, this allows for improved identification of phenotypic risks for genomes associated with cell-lines and embryos, which may further be used to develop medical tests and treatments based on selection and genome editing methods. In some embodiments, the identified phenotype risk scores may be used for human assisted reproduction, by allowing prospective parents more information in the selection of which embryos to implant. The present disclosure provides a method and system for employing the biology of crossover during reproduction to infer phenotypes from primary genomic sequence. By considering the primary source of short-term genomic variation to be crossover and recombination during reproduction, crossover segments called replicons are identified and employed to characterize organismal phenotypes. A replicon-based phenotype risk score is developed and extended to include small-structural variants that may be relevant to phenotypes when considered conditionally upon replicons. In some embodiments, a joint distribution is produced allowing the calculation of a weight distribution for each target phenotype conditioned on identification of replicons, or replicons combined with as small variants. Descriptions of processes for identifying replicon clusters, sometimes associated with haplotypes, and in developing models from a replicon segmentation of a genome are provided. Also provided are methods for developing such models from training sets including joint replicons and small variations. In some embodiments, phenotypes are derived from collections of distribution weights over phenotypes.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. As used herein, the term “embryo” generally refers to an unborn or unhatched offspring in the process of development. An embryo can refer to the product of fertilization or other approach of sexual reproduction as well as the products of asexual reproduction. In some embodiments, an embryo can be produced by fertilization of an egg with a sperm. In some embodiments, the embryo is produced by somatic cell nuclear transfer, parthenogenesis, androgenesis, or other asexual techniques. An embryo can refer to a zygote, a two-cell stage embryo, a four-cell stage embryo, an eight-cell stage embryo, a morula, or a blastocyst or blastula. An embryo can be produced in vivo or in vitro.

As used herein, the term “sequence read” refers to a DNA fragment for use in genetic or genomic sequencing. In some cases, sequence reads can be used to create sequencing libraries, which can be designed to interact with various sequencing platforms. In some cases, contigs, series of overlapping DNA fragments or reads, can be used to create sequencing libraries.

As used herein, the term “haplotype” refers to a set of DNA variations or polymorphisms that tend to be inherited together. A haplotype can be a combination of alleles. Alternatively, a haplotype can be a set of single nucleotide polymorphisms (SNPs) found on the same chromosome. A haplotype block, or haplo-block, is a region in which there is historically less genetic recombination. Haplo-blocks may have only a small number of distinct haplotypes.

### Genomic Samples

Embryonic, parental, and other genomes can be obtained through collection of genetic material. In some embodiments, the genetic material is obtained from blood, serum, plasma, sweat, hair, tears, urine, or tissue. Techniques for obtaining samples from a subject include, for example, obtaining samples by a mouth swab or a mouth wash, drawing blood, and obtaining a biopsy. In some cases, the genetic material is obtained from a biopsy, e.g., an embryo biopsy from the trophectoderm of a blastocyst. Isolating components of fluid or tissue samples (e.g., cells or RNA or DNA) may be accomplished using a variety of techniques. After the sample is obtained, it may be further processed to enrich for or purify genomic material.

If a sample (e.g., biopsy sample or culture sample) is treated to extract polynucleotides, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993, which is entirely incorporated herein by reference), with or without the use of an automated nucleic acid extractor, e.g., the Model 341 DNA Extractor available from Applied Biosystems (Foster City, Calif); (2) stationary phase adsorption methods (U.S. Pat. No. 5,234,809; Walsh et al., 1991, each of which is entirely incorporated herein by reference); and (3) salt-induced nucleic acid precipitation methods (Miller et al., (1988) which is entirely incorporated herein by reference), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628, which is entirely incorporated herein by reference). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724, which is entirely incorporated herein by reference. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g. absorbance of light at 260 nm, 280 nm, and a ratio of these) and detection of a label (e.g. fluorescent dyes and intercalating agents, such as SYBR green, SYBR blue, DAPI, propidium iodine, Hoechst stain, SYBR gold, ethidium bromide).

### Genomic Properties

Genomic properties, also referred to as genomic features, are characteristics of a genomic sequence, that may be aligned to a reference coordinate system for a particular species (e.g. hg19 for humans). The identification of genomic properties is often associated with a-priori scientific belief that such properties may be informative about organism phenotypes. Properties may be specifically identifiable as present or absent or may be represented as a weight or a probability of being present in the presence of uncertainty.

Mapping these properties to numeric values allows for inclusion in models that require numeric values as inputs. Without limitation, some such mappings include nucleotide (ACGT) at each genomic position, potentially encoded as hot-one features (FIG. 1A, 81); presence or type of variant from reference genome (e.g. hg19) at individual or conserved genomic positions (FIG. 1A, 80); presence of a deletion (FIG. 1A, 82); presence of an insertion (FIG. A1, 83); a replicon inheritance source (FIG. 1A, 84); an identified copy number variation (encoded by binary presence {0,1}) or a copy number count (FIG. 1A, 85). Replications may include much longer sequences of replicated DNA.

### Quantification of Phenotypes

A phenotype can be a discrete phenotype. A discrete or discontinuous phenotype is a phenotype that is controlled by one or a small number of genes. A discrete phenotype can be controlled by at least about 1, 2, 3, 4, 5, 6, 7, 8, 9, or 10 genes. Discreet phenotypes may have a small number of alleles and can have at least about 1, 2, 3, 4, 5, 6, 7, 8, 9, or 10 alleles. A non-limiting example of a discrete phenotype is the shape of pea seeds: smooth or wrinkled. Another non-limiting example of a discrete phenotype is the presence or absence of Type I diabetes.

A phenotype can be a continuous phenotype. A continuous phenotype is a phenotype that varies along a continuum in a population. Non-limiting examples of continuous phenotypes include height, blood pressure, reaction time, and learning ability.

Consider a phenotype of interest (ph). The phenotype may be discrete such as a binary variable representing, for example, the presence or absence of Type I diabetes at age 3, so that (ph∈{0,1}) where ph=0 represents the absence of the phenotype and ph=1 represents the presence of the phenotype.

Also consider a distribution of weights (W) across possible values of the phenotype (W|ph-> [symbol] ). These weights may be non-negative and normalized to 1, making them like probabilities ∀ph W (ph)≥0 and Σₚₕ W (ph)=1. However, weight need not be so normalized as per the weights in conditional random fields. In conditional random fields, weights are aggregated a normalized according to a “partition function” (Z) to form probabilities.

### Mapping Phenotype Risk Scores to a Figure of Merit

The process described herein can reduce complex genomic properties of each embryo in a collection, which may number in the millions, to a single figure of merit that may be used to rank each member of the collection. Methods and systems of the present disclosure may be applicable to the ranking of embryos generated during assisted fertility, by ranking each of embryo's associated genomic properties, so the most highly ranked embryo(s) can be prioritized for implantation.

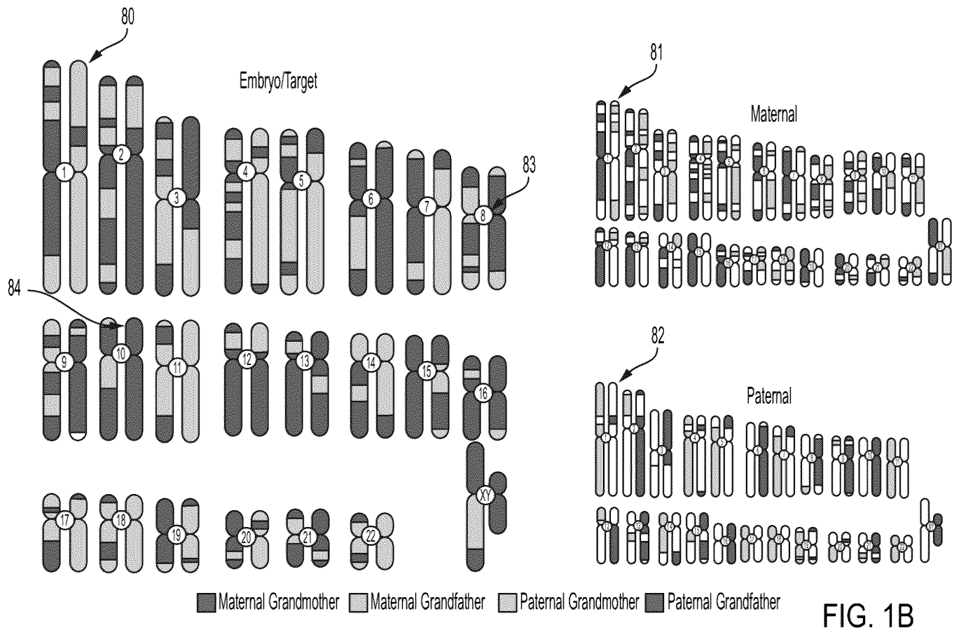

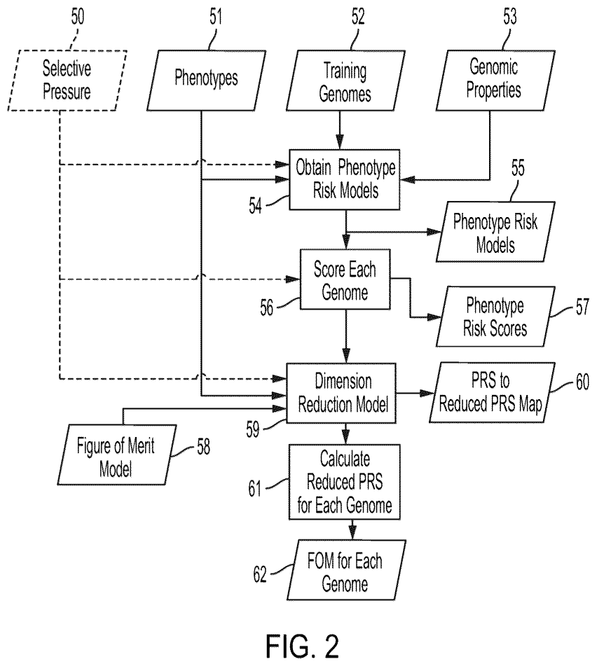

Considering a collection of numbered phenotypes Ph (FIG. 2-3, 51) where a particular phenotype enumerated with index i referred to as pᵢ such that pᵢ ∈Ph, and further a set of genomic properties G (FIG. 3, 11) which can be derived from a Specified Genome (FIG. 3, 10) along with a collection of risk models at least one for each phenotype pᵢ (FIG. 2-3, 55) which maps the genome into a real number (Wᵢ|G→ [symbol] ). An example of weighting is a Polygenic Risk Score (PRS) model (one type of Phenotype Risk Model generating one type of Phenotype Risk Score) that maps a collection of genomic variants to a probability of phenotype presence. For each genome G, Wᵢ can be used to map that genome to a collection of weights (FIG. 3, 12), one for each phenotype (FIG. 2, 57 and FIG. 3, 13). A PRS applies a linkage function that to maps the sum of the weights to statistical measure, such as probability of observation of the phenotype or odds ratio.

An example linkage function to one dimension Dᵢ|pᵢ→ [symbol] might be reduction in life expectation due to a phenotype, such as a disease. The single measure of impact of genome on expected longevity Vₐ(G) can be assessed as:

$${V}_{a} ( G ) = {\sum }_{i ∈ Phenotypes} {W}_{i} ( {p}_{i} \vert G ) {D}_{i} ( {p}_{i} ) / {\sum }_{i} {W}_{i} ( {p}_{i} \vert G )$$

In the case that the weighting Wᵢ is a probability, ΣᵢWᵢ(pᵢ|G)=1 and the value of the genome may be assessed [FIG. 3, 16] as

$${V}_{a} ( G ) = {\sum }_{i ∈ Phenotypes} {W}_{i} ( {p}_{i} \vert G ) {D}_{i} ( {p}_{i} )$$

In the case that separate per-phenotype measures for quality of life (medical and social measures are available) (FIG. 3, 60) quality of life can be accessed via a similar formula yielding V_{q}(G). Either of these figures of merit (FIG. 2-3, 58) may be used to rank a variety of genomes gⱼ∈G, or a composite V_{c}(gᵢ) may be developed. A natural measure for as V_{c}(gᵢ)=Vₐ(gᵢ)*V_{q}(gᵢ) measuring expected change in quality of life multiplied by life expectancy as a number of years experiencing the expected quality change (FIG. 3, 16). For example, this method may consider impacts that may be positive or negative, for example wherein a first trait such as disease resistance may increase quality adjusted life years, and a second trait such as disease susceptibility, may decrease it. Positive and negative traits may be incorporated by using the sign of a weight (not possible when weights are probabilities) or, alternatively, by adjusting the sign of the impact measurement, D to reflect positive or negative contributions. Embryos/cells/treatments/genomic modifications each with an expected genome g can be ranked according to the single value V_{c}(g)∈ [symbol] for investigation with the highest ranking investigated first. This method replaces a profusion of diverse phenotype risks, with a single more comprehensible measure of quality.

An alternative measure of quality might be considered proclivity to produce offspring, which in turn produce further offspring. The various phenotype weights that might be inferred from genomic information are intrinsically aggregated by the process of natural selection and assessed together via the historical impact on procreative fitness, which is in turn estimated by various models of mutation, recombination, and heritability; and inferred using observed allele frequencies across a species population, or across related diverged species (e.g. primates or eutherian mammals). For a particular reference genome for a species, such as hg19 (human) or GRCm39 (mouse), the selective pressure at each genomic position in the reference may be inferred across populations of individuals within each species (FIG. 2-3, 50). Methods such as phastCons and phyloP provide such a score at each genomic position for various collections of related organisms. A procreative phenotype score can by generated by aggregating the conservations scores at each genomic position associated with the phenotype (FIG. 3, 12).

For example, a polygenic risk score (PRS) is developed which creates a generalized linear mapping between variation at identified genomic position and the risk of observing a related phenotype; this score can be modified by multiplying every nonzero linear coefficient of the PRS, by a selective pressure measurement associated with the positions (FIG. 3, 12). This creates a weighted measurement of risk biased towards those positions with higher tendencies to be conserved. In some embodiments, every genomic position may be assigned a 0 or a 1 by assigning all genomic positions with phyloP conservation scores in the top fifth percentile (top 5% of all scores) the value 1, and the rest 0; then a PRS may be generated from only those positions assigned a 1. For human assessment, eutherian (placental) mammal phyloP scores mapped to hg19 might be used.

Four figures of merit are offered as examples of how collections of risk scores might be simplified by identifying expected impact of risk profile on that figure of merit. These figures of merit include the propensity to change expected lifespan (e.g., measuring years of life lost (YLL); ghdx.healthdata.org/ghd-results-tool) [Years of Life Lost (YLL): Global Burden of Disease Collaborative Network. Global Burden of Disease Study 2019 (GBD 2019) Burden by Risk 1990-2019. Seattle, United States of America: Institute for Health Metrics and Evaluation ORME), 2020]; the propensity to impact expected quality of life (QOL) [Quality Of Life (QOL): Ware, J. E., Gandek, B., Guyer, R. et al. Standardizing disease-specific quality of life measures across multiple chronic conditions: development and initial evaluation of the QOL Disease Impact Scale (QDIS®). Health Qual Life Outcomes 14, 84 (2016). doi.org/86/s12955-016-0483-x]; the propensity to change total quality of life, represented in some embodiments as the product of lifespan change and quality of life change (e.g., measuring disability adjusted life years (DALY)) [DALY: Disability Adjusted Life Years: Global Burden of Disease Collaborative Network. Global Burden of Disease Study 2019 (GBD 2019) Burden by Risk 1990-2019. Seattle, United States of America: Institute for Health Metrics and Evaluation (IHME), 2020 and Murray C J, Acharya A K. Understanding DALYs (disability-adjusted life years). J Health Econ. 1997 December; 16(6):703-30]; and the propensity to impact reproduction (measured by selective pressure e.g., using phyloP or PhastCons [phyloP and phastCons: Pollard K S, Hubisz M J, Rosenbloom K R, Siepel A. Detection of nonneutral substitution rates on mammalian phylogenies. Genome Res. 2010 January; 20(1):110-21]).

### Mapping Phenotype Risk Scores to Lower Complexity Trait Profiles

However, other simplifications can be provided that do not require reduction of genomes to a single score. There are numerous quantitative descriptions of complex psychological properties that are reduced to a small number of traits or types. This is useful when a simplified characterization of psychology is desired but sensitivity to differing situations is also a priority. Some of these are derived from defined characteristics (e.g., MTBI), while others may be derived from dimension reduction techniques applied to populations of scores, with reduced-dimensions (or clusters) being qualitatively characterized after quantitative discovery. For example, MTBI producers a characterization of personality in terms of four binary aspects, one of which is an opposed scale of introversion vs extroversion. Neither is given a linear contribution to a figure of merit, but rather may be differentially appropriate in different situations. For example, a worker in an isolated environment may be more successful if they have a personality that biases towards introversion (e.g., a researcher, or a scout), while one in an a more socially integrated environment (e.g., sales or marketing) may be more successful if their personality biases toward extroversion. Similarly, genomic predispositions for collections of phenotype biases in offspring or treatments may drive a preference that is conditionally dependent.

One example of a conditionally dependent preference is sex selection, sometimes called “family balancing” in IVF. Parents seeking a sex balance among offspring may prefer a female, if they have a male child, or a male child if they already have a female. Neither sex is unilaterally preferred, but sex may be a preference dependent on the context. By analogy, parents may prefer a child biased towards extroversion if they anticipate a strongly social environment, and a child biased towards introversion it they anticipate a more isolated one. Similarly, a genomic edit resulting in a collection of phenotypes that include inhibited insulin production, but resistance to leukemia, might provide an appropriate target for a bone marrow treatment when the implantation target is not responsible for insulin production, but might be unacceptable if the target tissue is responsible for insulin production.

A collection of Phenotype Risk Profiles (a distribution of phenotype risk scores) may be simplified by various techniques such as Principal Components Analysis, potentially followed by clustering techniques such as K-means or Hierarchical Agglomerative clustering, resulting in a smaller number of “types” [FIGS. 2 & 3, 60]. A similar technique involving the reduction of thousands of personality “terms” to sixteen and later 5 factors was used to develop the Big Five Personality trait model (Roccas, S.; Sagiv, L.; Schwartz, S. H.; Knafo, A. (2002). The Big Five Personality Factors and Personal Values. Personality and Social Psychology Bulletin, 28(6), 789-801. doi:10.1177/0146167202289008). Similarly, genomic predispositions embodied by phenotypic risk scores may be simplified to a smaller number of factors for suitability as a basis for treating disease, animal husbandry or selection of IVF generated embryos in assisted human reproduction.

Consider a collection of samples derived from a suitable population enumerated as n∈{1 . . . N} with each sample being represented by a corresponding set of genomic properties gₙ (where gₙ represents a collection of genomic properties for sample n) the collection of all such gₙ is referred to here as G (|G|=N). In addition. there a collection of phenotype models enumerates as i∈{1 . . . I} with each phenotype being represented as pᵢ. (where pᵢ represents a weight describing the risk of phenotype i∈I).

Using polygenic risk scores (PRS), maps can be created using gₙ→pᵢ, creating a weighting representing the risk of any phenotype for a sample given its collection of genomic properties:

wᵢ(gₙ)∈ [symbol] ^{k}

Where the weighting Wᵢ may be a single value representing a probability of a binary phenotype pᵢ (when k=1) or, alternatively, a distribution of probabilities across a continuous phenotype (e.g., height) such as those described by a Gaussian curve having a mean and a variance (k=2), or potentially a more complex distribution of weights requiring k parameters to define (FIG. 2, 54 and FIG. 3, 12). It is envisioned that weights are real values that may be positive or negative and a special case of weights are probabilities that are all greater than or equal to 0.0 and have a disjoint sum of 1.0.

In some embodiments, a univariate (k=1) estimate composed of a probability of a positive binary value, or a single central parameter (e.g. the mean for a Gaussian curve), may be considered.

Thus, for a collection of N genome samples G, Wᵢ defines a mapping from N samples to N collections of weights, each weight representing a propensity towards a phenotype i for sample n. This produces an N×I matrix values.

The complexity of this matrix may be reduced via any of a number of techniques, including PCA or Factor Analysis.

In PCA (Principal Component Analysis), the covariance matrix may represent an I×I matrix or covariances among phenotypes, estimates by aggregating across in the samples N. Using this covariance, the eigenvalues and vectors of the covariance matrix are calculated, and dimensional reductions (to dimension J where 0<J<I) are achieved by projecting any specific collection of inferred phenotypes i to the first J eigenvectors (e.g., the eigenvectors corresponding the eigenvalues of greatest magnitude), resulting in a collection of values of dimension J<I. The dimensions may be individually interrogated to characterize familiar properties of each dimension. This model may then be applied to genomic properties a novel sample to generate a representation of that novel sample which may in turn simplify comparison and preference ranking among other samples.

This reduced dimensionality representation may be presented as a representation or subset of a collection of genomic phenotype risks over I (FIG. 3, 13). Additionally, the representation (model) may be further simplified by identifying areas of high density (common collections of phenotypes) via clustering such as “k-means” clustering (simplifying when the number of clusters <J), and weights associated with each cluster (FIG. 3, 15) being presented to an evaluator for comparison among a variety of samples, or calibration of a single sample against a previous body of samples (FIG. 2, 60).

Many possible dimension reduction techniques are possible (FIG. 3, 14). The central idea is to simplify the analysis of a sample by mapping a collection of genomic properties (one collection per sample) to collection of phenotype weights (FIG. 3, 12) when reducing the complexity of the collection of phenotypes via dimension reduction (FIG. 3, 14), then presenting the reduced-complexity representation (FIG. 3, 15) (possible univariate, (FIG. 3, 16)) to evaluations to ease the comparison and possible selection of preferred samples.

Dimension reduction techniques include but are not limited to factor analysis, principal component analysis, independent component analysis, and t-Distributed Stochastic Neighbor Embedding (t-SNE, Van der Maaten, Laurens, and Geoffrey Hinton. “Visualizing data using t-SNE.” Journal of machine learning research 9.11 (2008). Any of these dimension reduction techniques can be followed by clustering to further simplify the underlying distributions, clustering techniques include but are not limited to gaussian mixtures, agglomerative clustering, spectral clustering, and k-means.

In cases where dimensional reduction is not directly amenable to explicit treatment of distributions (Risk Score distributions), the distribution can be treated empirically. This empirical treatment involves sampling risk phenotype values according to their weighting, and then providing applying dimensional reduction on the collection of samples that represents the distribution. For example, when phenotype weights are probabilities; empirical analysis customarily generates a sample frequency proportionately to the phenotype probability.

### Polygenic Risk Scores

Polygenic risk scores map genomic properties, relating these properties to disease risk. Diseases that can be mapped in this way include by are not limited to asthma, glaucoma, cancer, CV disease, CA disease, stroke, celiac disease, type 1 diabetes, arthritis, gout, Alzheimer's disease, autism, depression, and schizophrenia (Lambert, S. A., Gil, L., Jupp, S. et al. The Polygenic Score Catalog as an open database for reproducibility and systematic evaluation. Nat Genet 53, 420-425 (2021).

### Quality of Life Measures as a Figure of Merit

Quality of life measures attempt to convert assessable properties, such as the presence of disease, into mathematical features. The mathematical representation of Quality Of Life (QOL) facilitates analysis and comparison among phenotype alternatives. QOL measures may be limited to medical conditions. Alternatively, QOL measures can be broadened to include other factors (e.g., mental health). QOL models may be represented in economic units, such as impact on lifetime income, or more abstract measures of life quality. Some examples include models associated with AQoL-8D that attempts to assess QOL over 8 domains (www.aqol.com.au/, which is incorporated by reference herein in its entirety). There is also a disease specific subset particularly relevant to genomic phenotype analysis (www.aqol.com.au/index.php/aqol-current, which is incorporated by reference herein in its entirety) including items related to the presence and severity of arthritis, asthma, cancer, diabetes, and heart disease. The EQ-5D (including 5D-3L, 5D-Y)— provided by the European EuroQol Research Foundation (eurowl.org/, which is incorporated by reference herein in its entirety); SF-36, which maps phenotypic traits directly to quality-adjusted life-years and is a subset the SF-12 available from the RAND corporation (Jenkinson C, Layte R, Jenkinson D, Lawrence K, Petersen S, Paice C, Stradling J. A shorter form health survey: can the SF-12 replicate results from the SF-36 in longitudinal studies? Journal of Public Health Medicine. 1997, 19 (2): 179-186), which is incorporated by reference herein in its entirety); and HRQOL-14 from the Unites States CDC (www.cdc.gov/hrgol/hrgo114_measure.htm, which is incorporated by reference herein in its entirety) are additional exemplary models associated with QOL measurements.

Another approach to QOL is to assess subjective wellbeing (e.g. happiness), using instruments that attempt to map happiness to mathematical values including but not limited to the Oxford Happiness Inventory (Argyle and Hill), the Panas Scale (Watson, Clark, Tellegen) and PNAS-Gen (Watson, D., Clark, L. A., Tellegen, A. (1988), each of which is incorporated by reference herein in its entirety). Development and validation of brief measures of positive and negative affect: The PANAS scales. Journal of Personality and Social Psychology, (54), 1063-1070, which is incorporated by reference herein in its entirety.). OECD Guidelines on Measuring Subjective Well-being. (www.oecd.org/statistics/oecd-guidelines-on-measuring-subjective-well-being-9789264191655-en.htm, which is incorporated by reference herein in its entirety). Summarizing health-related quality of life (HRQOL): development and testing of a one-factor model. Shaoman Yin, Rashid Njai, Lawrence Barker, Paul Z. Siegel, and Youlian Liao, which is incorporated by reference herein in its entirety.

While many questions in a QOL survey can map directly to disease phenotypes that have existing PRS models, others can require development of new PRS, inference of expected answers, or resultant measures with some PRS models. Causal models include various structural equation models and observational models that are restricted to causal criteria such as Front Door and Back Door criteria (Pearl, Judea; Causality: Models, Reasoning and Inference; second edition, 2009; isbn 052189560X; Cambridge University Pres; USA).

### Description of Process

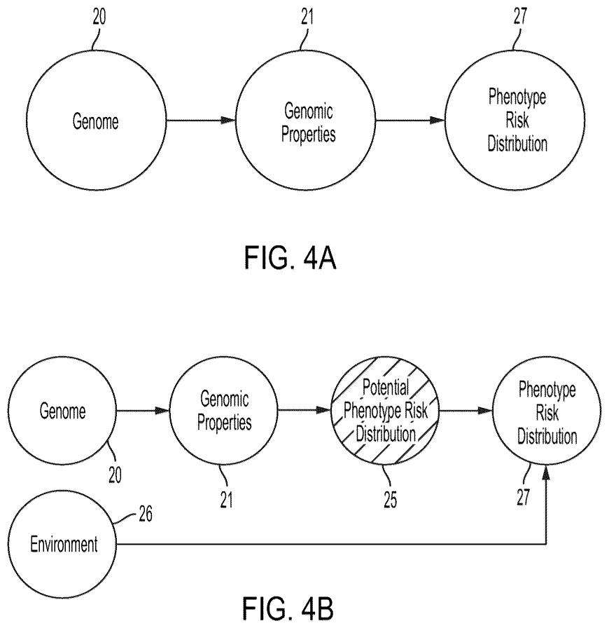

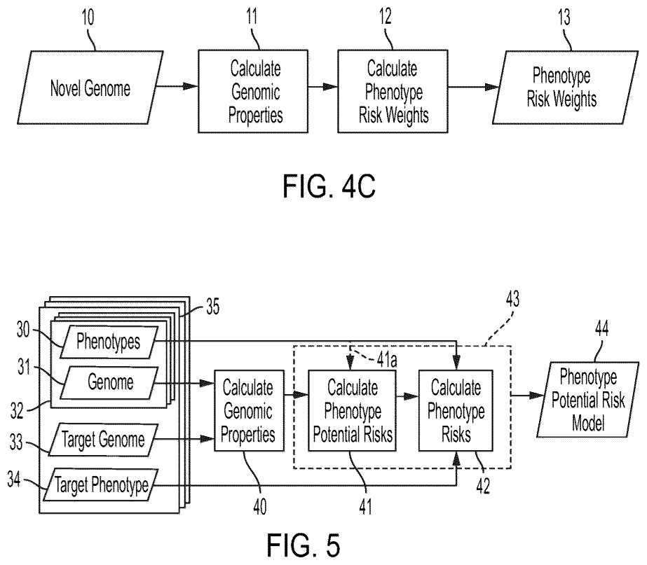

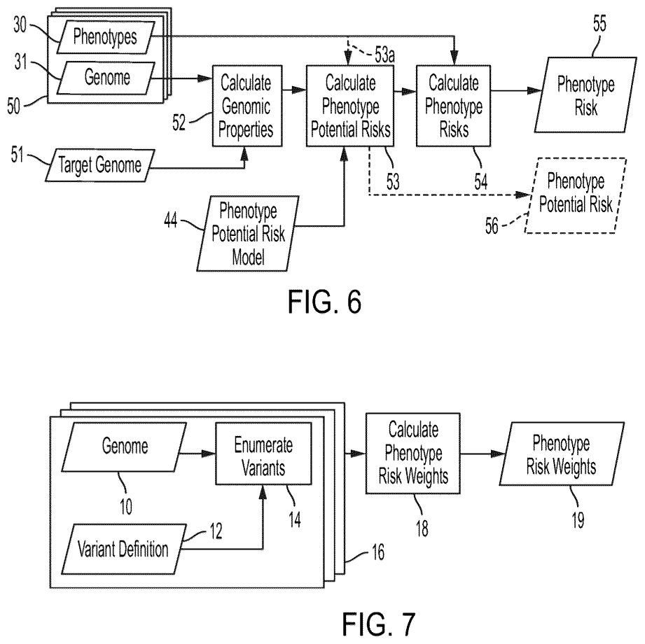

The prediction process accepts a trained phenotype potential risk model (FIG. 5-6, 44) a corresponding collection (FIG. 6, 50) of related genomes (FIG. 6, 31) and phenotypes (FIG. 6, 30) and a target genome (FIG. 6, 51). The result is a target phenotype risk distribution (FIG. 6, 55) for a phenotype of interest that may not yet be manifest in the target organism. In some embodiments of the model a target potential phenotype risk distribution (FIG. 6, 56) may be produced which describes the target phenotype risk distribution for each combination of related phenotypes (FIG. 6, 30). In some embodiments, the calculation of phenotype risk may proceed directly without the explicit calculation of a potential phenotype risk distribution, though this potential distribution is still implicitly defined by the model. An explicit potential phenotype risk distribution may be of particular value in considering differing environments in which a target may exist or be developed. For example, an engineered cell type with a particular genome may grow in a desired fashion in an oxygen rich environment but fail to grow in an oxygen poor one. Similarly, a particular embryo may have extraordinary propensity to flourish to a healthy weight in a relatively low-calorie environment (a first phenotype) but be at substantially elevated risk for adult onset Type 2 Diabetes in a high-calorie environment (a second phenotype). A risk distribution can contribute a positive expected value, a negative expected value, or a zero expected value.

An embodiment of the present disclosure can include, but is not limited to, a logistical model, separate models for each combination of related phenotypes, neural network/deep learning models, single tree models such as CART, random forest models, support vector machines, generalized linear models, or logistic regressions.

In addition, the logistical model method of incorporating related phenotypes is not restricted to inference of child phenotypes from two parents. Larger collections of phenotypes may provide for extended demographic cohorts including families, tribes, or regional populations. In cases where the observation of a trait may be depend on relatives' phenotypes but NOT some relatives' genotypes, the genomic properties for each genome in a related set (FIG. 6, 50) may vary for each individual, for example, masking out all genomic traits for uncles or cousins, while allowing some genomic properties for parents or the target. This may be particularly useful when the phenotype risk distributions of the target are conditionally independent of related genomes, when conditioned on a target genome. Furthermore, this method may also extend to predicting useful traits in livestock animals (such as weight in bovines) or assessing expected phenotypes for genetically edited (e.g. via CRISPR-Cas9) variants of a source cell type used in the development of medical treatments and remediations. Additionally, the phenotype element of the model (FIG. 6, 30), along with the use of parental or related biological properties (biological phenotypes) are a proxy for environmental conditions that may not be defined.

### Developing Models

Each model map represents a transformation from a collection of inputs to an output. In various embodiments, such models may have parameters which are determined through the use of a training set, then applied to novel data outside the training set. In various embodiments, parameters are adjusted so that the training data when applied to parameterized model, most closely approximates that desired output, subject to complexity constraints. In some embodiments, the output may be a figure of merit, or a reduced representation that is more concise than the collection of phenotype risk Scores for each genome.

In FIG. 2, a collection of training genomes (FIG. 2, 52) is used to derive a corresponding distribution for each collection genomic properties (FIG. 2, 53), providing one distribution for each property in the collection for each genomic property. Thus, each genome is represented by a collection of associated genomic properties. Properties are selected so they can serve as inputs to each phenotype risk model (FIG. 2, 54). Risk models can be collected from an outside source or can be generated during development and provided as output (FIG. 2, 55), corresponding to input in FIG. 3, 55. The set of distributions of genomic properties for each training genome is applied to each phenotype risk model (FIG. 2, 56) to generate a phenotype score distribution for each phenotype associated with each genome, generating a collection of phenotype risk scores (FIG. 2, 57) for each genome, one per phenotype. This may require aggregating weights or probabilities of a particular phenotype state (e.g. severities of diabetes) across distributions of genomic properties and PRM to generate distributions of risk for each phenotype. A dimension reduction model is then generated (FIG. 2, 59) by any of a number of reduction techniques to create a reduced PRS map (FIG. 2, 60). An intermediate step may then require calculating a reduced complexity distribution of weights (FIG. 2, 61) by applying the DRM (FIG. 2, 59) to each of phenotype risk scores (FIG. 2, 56) for each training genome to generate a simpler predictive representation from those scores. The DRM is a model whose parameters are chosen so as to minimize complexity, and if selected, improve the expected prediction of the FOM for the organism associated with the corresponding genome.

In developing PRS and GWAS, a body of genome-phenotype pairs are combined to calibrate statistical models (subject to complexity constraints) by adjusting model parameters to minimize predictive error. Once stopping criteria are met, the parameters are fixed, and the model is available to make predictions about the likely eventual phenotype risk conditioned on a genome.

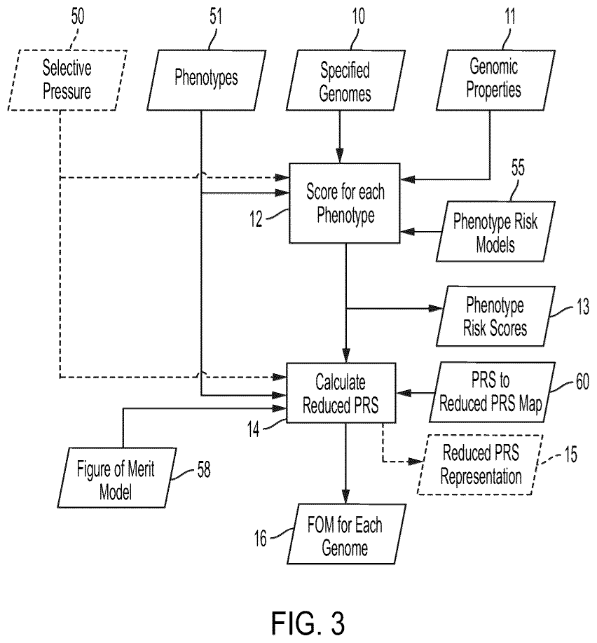

Broadly, the goal of the phenotype risk model is to use an existing set of phenotypes and genotypes for target and related organisms, to build a statistical model that allows for the prediction or target phenotype risk from related phenotypes and target genotypes alone (FIG. 4B). The statistical model is characterized by a set of parameters, which may begin as random values and are refined via training so as to minimize a combination of predictive error over the training set, and model complexity. This model is then applied to a novel target genome (along with related phenotypes an genotypes) to estimate the risk of that an organism that possesses the target genome will eventually develop the target phenotype.

In some embodiments, model development begins with a collection of training data (FIG. 5, 35), each training data element contains: a target genome (FIG. 5, 33), a collection of target phenotypes (FIG. 5, 34) associated with the organism possessing that target genome, a collection (FIG. 5, 32) of related genomes (FIG. 5, 31) and phenotypes (FIG. 5, 30) with one complete set of phenotypes for each related genome. Each genome in the collection of training data is mapped into a corresponding set of numeric Genomic Properties (FIG. 5, 40). An initial phenotype risk model, perhaps as simple as a generalized linear model (GLM, one example being Logistic Regression) is then applied to the collection of training genomes and target genomes (genomes represented by their corresponding genomic properties) to assess the genomic potential phenotype risk of the target (FIG. 5, 41). This potential phenotype risk is a distribution representing the genomic potential risk of the target, without consideration of the target's actual developmental environment (FIG. 4B, 25). It is considered as an additional possibility that this risk itself may be influenced by the environment, for example, via environment-induced epigenomic changes that might, in turn, influence predisposition towards primary sequences in offspring. Epigenetic mechanisms that may signal such an influence include PMDR9 binding and DNA methylation. Some model embodiments may ignore this effect, while others may take it into account, the impact of inclusion is represented as a dashed line in FIG. 5, 41a, representing an envisioned but optional component. In constructing a model, the phenotype potential risks are then combined with environmental factors to produce a risk distribution for the collection of target phenotypes (FIG. 5, 42). There can be at least about 1, 2, 3, 4, 5, 6, 7, 8, 9, 10, 15, 20, 30, 40, 50, 60, 70, 80, 90, 100, 150, 200, 300, 400, 500, 600, 700, 800, 900, 1000 or more target phenotype values. There can be at most about 1000, 900, 800, 700, 600, 500, 400, 300, 200, 150, 100, 90, 80, 70, 60, 50, 40, 30, 20, 15, 10, 9, 8, 7, 6, 5, 4, 3, 2, or 1 target phenotype values. This risk distribution is compared to the actual collection of target phenotypes (FIG. 5, 34) and the parameters of the model adjusted to reduce discrepancy between the prediction and the target phenotype, subject to complexity constraints. Many techniques can perform this reduction, (e.g., gradient descent, regularized gradient descent, binary and gridded search, or evolutionary search). The discrepancy reduction process is then repeated (FIG. 5, 43) iterating across the collection of training elements (FIG. 5, 35) until a stopping point is reached. Some examples of stopping points include but are not limited to a minimal reduction in discrepancy between prediction and target phenotypes across the training set or the attainment of a particular absolute discrepancy (error) level. When the stopping criteria are met and training ceases, the current parameters form the core of the potential phenotype risk model.

In some embodiments, the set of phenotypes may be much larger than the target phenotype. These represent features other than a trait that may impact that trait, for example binary presence of parental obesity at a particular age ({0,1}) may be informative as to risk of Type 2 Diabetes being manifest by developing target organism by adulthood (for example, another binary variable {0,1}).

In some embodiments, a variety of environmental factors may be included in the prediction of phenotypes. Such environmental factors may include, but are not limited to, environmental or developmental features that may (directly, or indirectly) influence the manifestation of an organismal property (traditional phenotype) from a genomic risk or predisposition. Such environmental features might include an exposure to toxic chemicals like asbestos, availability of medical care, socioeconomic status (possibly as described by presence of poverty), or exposure to familial famine/starvation during adolescence. All of these factors may be predictive of the risk profile for a traditional target phenotype such as lung cancer, diabetes, adult height, weight at a specific age, or hypertension.

One non-limiting example of the use of a method of the disclosure is in assessing embryos generated by two known parents for human assisted reproduction. In this case the training data might consist of trios of one offspring (as the target) and two parents. The genomes of the parents as well as their phenotypes are known or inferred (e.g., UK Biobank, Sudlow C, Gallacher J, Allen N, Beral V, Burton P, Danesh J, et al. (2015) UK Biobank: An Open Access Resource for Identifying the Causes of a Wide Range of Complex Diseases of Middle and Old Age. PLoS Med 12(3): e1001779.see also www.ukbiobank.ac.uk), as is the target genome and target phenotype.

The phenotype potential risk model accepts genotype properties from the target and its relatives and can produce a distribution over related phenotypes and target phenotypes. Combining this distribution with related phenotypes produces a distribution over target phenotypes. In some embodiments, this process is combined into a single calculation and no separate phenotype potential risk analysis may be produced.

### Incorporating Underlying Statistical Relationships

One observation is that at short time scales, survivable organismal variety derives primarily from offspring genomic variation induced by crossover during reproduction, rather than de-novo mutation (variation not shared with either parent). The methods described herein seek to utilize this variation as the primary predictor of phenotype, with de-novo mutation playing a secondary role. In some embodiments, this is applied toward estimating single-generation phenotype inheritance of an embryonic genome from parental genomes in the course of assisted reproduction. A further specification of this application may be in embryonic selection and ranking for human IVF.

De-novo mutation (not inherited from either parent) may also play an appreciable role in embryonic phenotype. However, methods and systems of the present disclosure may not analyze de-novo mutation when conditioned on replicons. The methods and systems of the present disclosure may allow that a mutation in one replicon may have an important phenotypic role, for example disrupting a transcription factor binding motif leading to variation in phenotypically relevant gene transcription, while the same mutation in a different replicon (with no transcription factor binding site) at the same genomic coordinates may have little phenotypical impact. This may be especially important when the phenotype represents predisposition to a human disease.

Some genomic methods for inferring phenotype risk scores such as polygenic risk scoring, may derive from a genome-driven relationship, first extracting a collection of genomic properties from each particular genome, and them estimating the risk of observing a phenotype in an organism with the corresponding genome, directly from that collection of genomic properties (FIG. 4A). In such a model, the relatively identical genomes may yield a relatively identical collection of genomic properties (as in the case of monozygotic twins). Similarly, identical collections of genomic properties associated with separate organisms may yield identical phenotype risk distribution estimates, regardless of the environment in which the organisms develop. The proposed work expands this model by incorporating specific consideration of the environment (FIG. 4B). In the proposed framework, genomes and genomic properties may be calculated in a similar fashion; however, the genomic properties are used to identify a potential phenotype risk distribution, which represents contributions to a latent and potentially unobservable predisposition towards risk. Conceptually, this potential represents an unknown genomic system or process that may respond differently to differing environments, producing different organismal phenotypes. This process is represented as a model that accepts inputs pertaining to the environment and produces a risk distribution. In some embodiments, the potential risk distribution might be considered a multivariate probability distribution that includes risk distributions and environmental properties as variables, and that can be used to generate a specific risk distribution when conditioned on known values for those environmental variables. FIG. 4A-B may be considered in one instance as probabilistic graphical models, though that is not indented as a limitation.

In some embodiments, a collection (G) of N genomes (N=|G|) may be enumerated as gₙ ∈G. A set of genomic properties is selected to represent each genome as a collection of numbers. For a specific collection of properties (GP) the collection of property values associated with a particular genome g h can be identified as GP (gₙ)=gpₙ or by mapping gₙ→gpₙ. A training set used to build a model represents a known combination of genotypes G along with an associated collection of phenotypes (PH) where the organism n is associated with genome gₙ and a specific collection of phenotypes phₙ. A model accepts gpₙ and produces an estimated distribution over one or more phₙ. Such models are composed of parameters which are adjusted to minimize a combination of complexity and error in the estimated distribution. One specific embodiment of a model is the family of generalized linear models, for example, logistic regression. For a considering a single binary phenotype (phₙ ∈{0,1}), such a model for predicting probabilities (P) may be represented as

$$P ( {ph}_{n} = 1 ) = Link ( {\sum }_{n ∈ \vert GP \vert} M \cdot {gp}_{n} ) \\ P ( {ph}_{n} = 0 ) = 1 - P ( {ph}_{n} = 1 )$$

Where one commonly used link function is the logistic function:

$$Link ( x ) = \frac{L}{1 + {e}^{- k ( x - {x}_{0} )}}$$

which simplifies to the following equation for trivial parameters, L=1, k=1, x₀=0

$$Link ( x ) = \frac{1}{1 + {e}^{- x}}$$

Where the link function is a logistic map, mapping takes all real values onto the range (0,1). Many more sophisticated techniques for creating such a map gpₙ→Distribution(phₙ) are include but are not limited to neutral networks, deep learning systems, random forests and CART maps. One technique for refining the parameters in these models involves initializing parameters with central values from the distributions in a training set, and then adjusting parameters in a way that reduces the net predictive error, potentially constrained by complexity limitations. This technique is referred to as gradient descent, and may be iterated until a minimum predictive error is found, or another iteration stopping criteria is met.

A generalized linear model serves as one useful embodiment for considering a genomic assessment process in FIG. 4C. In this process, a single genome gₙ is analyzed, by first mapping it to a collection of numeric genomic properties gpₙ. A model trained as described above is then used to calculate phenotype risk weights. Some embodiments of this may be considered as:

gpₙ→Distrib(phₙ)

where output Distrib(phₙ) is represented in FIG. 4C, 13. In the simple case that that there is one binary phenotype this distribution can be determined in the final step, as the probability distribution

gpₙ→P(phₙ=1)

with

$$P ( {ph}_{n} = 1 ) = Link ( {\sum }_{n ∈ \vert GP \vert} M \cdot {gp}_{n} )$$

where M is a model in the form of a matrix, with matrix elements serving as model parameters, that is derived from training on a previously identified training set.

### Replicon Determination

In some embodiments, replicons may be identified by identifying all crossover points in a known population and segmenting the genome according to the union of all such crossover points. For a given coordinate segment, defined as the contiguous region between adjacent crossovers, all uniquely observed nucleotide sequences can be listed. If small variants generate an unacceptably large number of uniquely observed nucleotide sequences, they may be clustered into a smaller number of clusters, each cluster associated with a unique replicon ID. This allows for minor variations on a single sequence to be treated as corresponding to the same replicon. In some embodiments, this clustering may be performed so as to maximize complexity-limited predictive power over phenotypes, especially during PRS training. In some embodiments, approaches may consider linkage disequilibrium, or alternatively down-weight variations at sites that are not under longer-term selective pressure, or variations at locations known a-priori to be less likely to participate in genomic function.

In some embodiments, replicons may be associated with demographic haplotypes. These haplotypes serve as a curated source of replicon clusters as defined above and are considered significant in the definition of demographic characteristics.

In some embodiments, replicons may be clustered using clustering parameters generated the context of model training so as to yield the clusters most likely to be informative about phenotypes. This can be accomplished by gradient methods on cluster variation parameters so as to maximize the complexity-limited probability of the observed training set.

### Methods for Analysis

In some embodiments, a PRS method is performed. A PRS might proceed by gathering a collection of genomes and corresponding phenotypes. Genomic properties that define variants are quantified. The genomic properties are used to map features of each genome to a collection of numeric properties for further analysis. One such mapping is the identification of specific variants. In some embodiments, for phenotypes y, N individuals, M genotypes, Z is N×M matrix of standardized (columns are scaled to 0 mean and unit variance), β is a vector of effect sizes (one per genotype), ∈ is a vector of environmental effects (noise). Further, in a Bayesian setting a non-informative Jefferies prior on the residual variance σ² can be set or an empirical estimator \hat{σ} can be derived via methods such as gradient descent to maximize likelihood.

Standardization is performed column-wise, if there is only a single numerical phenotype representation then there is only a single mean and standard deviation

$${y}_{std} = \frac{( y - μ ( y ) )}{σ ( y )}$$

Where μ(y) is the column-wise mean value for y and σ(y) is the column-wise standard deviation.

y=Zβ+∈,∈˜N(σ,σ²I)

and an estimator for then effect size \hat{β} can be calculated as

$$\hat{β} = \frac{{Z}^{T} {y}_{std}}{N}$$

leaving a phenotype likelihood as Πᵢ\hat{∈}(yᵢ−(Z\hat{β})ᵢ) or as a loglikelihood

$${\sum }_{i} log ( \hat{ϵ} ( {y}_{i} - {( Z \hat{β} )}_{i} ) )$$

More sophisticated generalized linear models can be used, introducing a link function representing y=Link (Zβ+∈₁)+∈₂. One link function can be the logistic function

$${Link}_{\{ μ , k \}} ( x ) = \frac{1}{1 + {e}^{- k ( x - μ )}}$$

where μ and k become model parameters that may be fit by various approaches including via maximum likelihood, MAP or expectation.

In some embodiments, a numeric phenotype matrix y based on the presence {1} or absence {0} of the target phenotype can be constructed, then adjust to standard form. If there is a single phenotype the matrix may have a single column and one row for each genome:

$$y = Z β + ϵ , ϵ \sim N ( o , {σ}^{2} I ) , \hat{β} = \frac{{Z}^{T} y}{N}$$

Once a model has been identified (e.g. link function and all model parameters), parameters are calibrated using a training set via a number of methods, as described elsewhere herein, to generate a trained model. This trained model serves as a map allowing a calculation from any collection of consistent genomic parameters to a phenotype risk weight. This risk weight may be converted to a probabilistic representation of phenotype value. In an alternative embodiment, an adaptive model may be used, for example, allowing the number of parameters to increase as the informative power of the training data increases.

Numerous methods can be used for training and applying numerical genomic properties to numerical phenotype risk distributions, some of which provide complexity limitations to improve model generalizability. These include, but are not limited to, generalized linear models, CART trees, random forests, gradient boosted trees/forests, neutral networks, and deep learning systems. Selecting the correct set of genomic features may be important, so that a trained model is likely to generate an accurate map.

The present disclosure provides methods and systems that may focus on the idea that replicons are important genomic features in identifying phenotypic variation, especially at short durations such as the offspring of a single set of parents.

### Basic Replicon Analysis

In some embodiments, a replicon definition is obtained and applied to each genome in of a collection of training set elements, each element consisting of a genome and a corresponding set of phenotypes. The replicon definition allows for the mapping of identified replicons to distributions of numerical values. The collection of distributions over replicon clusters is used to calculate phenotypic risk weights by application of single replicon values to a model or via aggregating weights for replicon distributions across individual replicon (element) to form an expectation, for example:

$$E [ Score , element ] = ( \frac{{\sum }_{r ∈ Replicon} ( W ( r , element ) \cdot Score ( r ) )}{{\sum }_{r ∈ Replicon} W ( r , element )} )$$

$$E [ Score ] = {\sum }_{element ∈ Genome} E [ Score , element ]$$

This risk score is then compared to the corresponding phenotype in the training set and the difference used to generate a gradient descent training of PRS model parameters. Once this model is trained and fixed, a novel genome may similarly be enumerated into a distribution across replicons. This distribution over replicons can then be applied to the PRS component of the model to calculate a distribution over phenotype risk weights. This distribution over PRW may be further collapsed into more simplified expectations for comparison with other risk scores, or collections of risk scores.

Additionally, small genomic variation may be combined with replicon variation to further refine phenotype risk scores. A conceptual framework for this method is that variations at specific genomic positions may carry significant phenotypic risk impact for one replicon, perhaps altering an important nucleotide in a transcription factor binding site, while that same variation in another replicon (perhaps one without that site, where the site was shifted, or its function moves to another genomic location) may have limited phenotypic risk impact.

### Advanced Replicon Analysis

In understanding an embodiment that combines replicon definition with variant definition, a machine learning property called regularization may be considered. Regularization places a penalty on model complexity that may be included as part of a model's error term during training. Thus, during the training of a model, the error gradient favors model parameters that are both accurate and simple (parameters of low magnitude may be a proxy for simplicity). This is desirable as models of lower complexity with similar training errors, may generalize better to non-test-set examples than models of higher complexity when trained on the same training sets (i.e., Ockham's razor). As the amount and diversity of training data grows, the amount of allowed complexity acceptable by the model can grow commensurately, justified by the additional data. In a regularized model, a single replicon covariate that is very informative about polygenic risk may be favored over a collection of individual variations that together are similarly informative (or in some cases, are more informative). As the amount of training data grows, improvements in polygenic risk prediction from individual variants, above those for coarser replicons, may drive an increase in model complexity/parameters that in turn assigns non-zero weight to individual variations. Some embodiments of this joint model is described below.

Consider the model referenced in this document:

y=Zβ+∈,∈˜N(σ,σ²I).

The target phenotypes and number of individuals remains the same, as phenotypes y, and N individuals. However, the M enumerated variations (genotypes) are replaced with an aggregate of at least one of the enumerated replicons and an appropriately sized collection of enumerated variants. The total number of variables M is now the sum of the number of enumerated replicons (Mᵣ) and enumerated variants (Mᵥ) such that M=Mᵣ+Mᵥ and similarly |β|=M, with β coefficients corresponding to each of these enumerations. However, to favor more parsimonious (and generalizable) attribution of model complexity to more predictive coefficients, a regularization term may be added. While there are many embodiments of regularization this example considers a L₁ style complexity as it is: often amenable to efficient convex optimization (unlike Lₙ regularization where 0<n<1), and tends to drive model parameters to a magnitude of 0 when they are less informative, making them more robust to noise (unlike L₂ regularization). (The Lₙ norm may be an aggregate of the form Lₙ(β)=Σᵢ(|βᵢ|)ⁿ. Here |β| is the L₁ magnitude of all coefficients |β|=Σᵢ|βᵢ|).

Identifying values for the model \hat{β}, \hat{α} now takes the form of adjusting the model training structure to

Err_{β,α}(y)=(y−ŷ)²=(y−(Zβ+α|β|+∈))²

\hat{β},\hat{α}=argmin_{β,α}(Err_{β,α}(y))

Where α is called a hyperparameter; the value of a hyperparameter may be determined empirically during model training over a set of training data represented by phenotypes y and combined replicon and variation values Z.

Once values for \hat{β} are obtained, prediction on novel genomes proceeds similarly as before with

y=Z\hat{β}+∈

where the replicon and variant definitions are the same as those used during training, and the enumeration of replicons Zᵣₑₚ and variants Zᵥₐᵣ in predicting PRS for a genome (Z=[−Zᵣₑₚ,Zᵥₐᵣ]) The prediction can be applied to a single genome (thus N=1, and the collection has just one element), or it can be applied to multiple genomes. The variant and replicon enumeration remains separate (\hat{β}=[\hat{β}ᵣₑₚ, \hat{β}ᵥₐᵣ]), so the equation may be more easily understood as

y=Zᵣₑₚ\hat{β}ᵣₑₚ+Zᵥₐᵣ\hat{β}ᵥₐᵣ+∈

Calculation of this equation predicts a distribution of phenotype risk weights across phenotype quantification, y.

These methods may also be used to map replicon and variant definitions (Z) to phenotype distribution predictions (y). Some of these methods include generalized linear models, taking a form of a link function applied to a linear model, in one form y=Link(Z\hat{β}+∈₁)+∈₂ where the link function may identify the logistic function, exponential, polynomial, identity, etc. Decision tree models such as CART, classification and regression trees, are useful for modeling intelligible, but non-linear, relationships, and random forest methods represent some of the most powerful contemporary non-linear estimators, but often suffer from difficulty in intelligibility. Deep Learning/Neural Network methods learn powerful predictive functions, but like random forest methods, can also suffer from difficulty in intelligibility. Another possible method includes using stratified analysis where variants are only analyzed within the context of a specific replicon or distribution of replicons, analogous to a conditional random field.

In some embodiments, a feature of blended replicon/variant analysis is regularization, which biases the model to explain phenotypes in a parsimonious way, employing the implicit broad correlations inherent in replicon structure to model phenotypes when possible and using smaller variations to explain differences between replicons, when they are warranted.

### Trained Algorithms

Once the model is fully built, the algorithm can be used to rank genomes according to figures of merit. For example, the trained algorithm may be used to determine quantitative measures of expected embryonic future health, during the course of IVF implementation decisions. The trained algorithm may be configured to identify future embryonic health with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 21, at least about 22, at least about 23, at least about 24, at least about 25, at least about 26, at least about 27, at least about 28, at least about 29, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, at least about 600, at least about 700, at least about 800, at least about 900, at least about 1000, or more than about 1,000 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise genomic or phenotypic data. For example, an input variable may comprise whether or not a biological parent has Type II diabetes.

The trained algorithm may comprise a classifier, such that each of the one or more output values identifies probabilities of discrete classifications, or otherwise indicates one of a fixed number of possible values (e.g., a linear classifier, a logistic regression classifier, etc.) indicating a classification of the biological sample by the classifier. The trained algorithm may comprise a binary classifier, such that each of the one or more output values comprises one of two values (e.g., {0, 1}, {positive, negative}, or {high-risk, low-risk}) indicating a classification of the biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Some descriptive labels may be mapped to numerical values, for example, by mapping “positive” to 1 and “negative” to 0. Biological samples may be derived from whole cells, fractional cells, or cell-free media derived from, for example, embryo incubation media, blood distillate, or amniotic fluid.

Some of the output values may comprise numerical values, such as binary, integer, or continuous values. Such binary output values may comprise, for example, {0, 1},{positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the pregnancy-related state of the subject. Some numerical values may be mapped to descriptive labels, for example, by mapping 1 to “positive” and 0 to “negative.”

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, at least about 500, at least about 600, at least about 700, at least about 800, at least about 900, at least about 1,000, at least about 2,000, at least about 3,000, at least about 4,000, at least about 5,000, at least about 6,000, at least about 7,000, at least about 8,000, at least about 9,000, at least about 10,000, at least about 50,000, at least about 100,000, at least about 500,000, at least about 1,000,000, at least about 10,000,000, at least about 100,000,000, or at least about 1,000,000,000 independent training samples.

While the trained algorithm is initially trained, a subset of the inputs may be identified as most influential or most important to be included for making high-quality classifications. Alternatively, after the trained algorithm is initially trained, a subset of the inputs may be identified as most influential or most important to be included for making high-quality classifications. For example, a subset of the studied genotypes/phenotypes may be identified as most influential or most important to be included for making high-quality classifications or identifications of embryonic ranking. For example, if training with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

Computer Systems

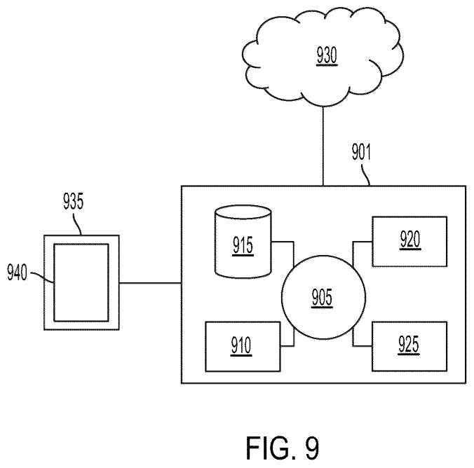

The present disclosure provides computer systems that can be programmed to implement methods of the disclosure. FIG. 9 shows an exemplary computer system 901 that is programmed or otherwise configured, but not limited to, for example, (i) train and test a trained algorithm, (ii) use the trained algorithm to process data to determine the future health of an embryo, (iii) determine a quantitative measure indicative of the future health of an embryo, and/or (iv) electronically output a report that is indicative of the future health of an embryo.

In some embodiments, the systems and methods of the present disclosure utilize algorithms capable of training. In other embodiments, however, the system and methods of the present disclosure may use a pre-trained algorithm.

The computer system 901 can regulate various aspects of analysis, calculation, and generation of the present disclosure, such as, for example, (i) training and testing a trained algorithm, (ii) using the trained algorithm to process data to determine the future health of an embryo, (iii) determining a quantitative measure indicative of the future health of an embryo, and (iv) electronically outputting a report that is indicative of the future health of an embryo. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet.

The network 930 in some cases is a telecommunication and/or data network. The network 930 can include one or more computer servers, which can enable distributed computing, such as cloud computing. For example, one or more computer servers may enable cloud computing over the network 930 (“the cloud”) to perform various aspects of analysis, calculation, and generation of the present disclosure, such as, for example, (i) training and testing a trained algorithm, (ii) using the trained algorithm to process data to determine the future health of an embryo, (iii) determining a quantitative measure indicative of the future health of an embryo, and (iv) electronically outputting a report that is indicative of the future health of an embryo. Such cloud computing may be provided by cloud computing platforms such as, for example, Amazon Web Services (AWS), Microsoft Azure, Google Cloud Platform, and IBM cloud. The network 930, in some cases with the aid of the computer system 901, can implement a peer-to-peer network, which may enable devices coupled to the computer system 901 to behave as a client or a server.

The CPU 905 may comprise one or more computer processors and/or one or more graphics processing units (GPUs). The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC). Without limitation, in some cases the circuit is derived from a specialized graphics processing unit or accelerator commonly used for machine learning applications.

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine-readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, quantum mechanical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, (i) a visual display indicative of training and testing of a trained algorithm, (ii) a visual display of data indicating the future health of an embryo, (iii) a quantitative measure of the data indicating the future health of an embryo, or (iv) an electronic report of the future health of an embryo. Examples of UIs include, without limitation, a graphical user interface (GUI), a web-based user interface, or a printer/printed report.

Methods of the present disclosure can be implemented by way of one or more algorithms. An algorithm can be implemented by way of software upon execution by the central processing unit 905.

### EXAMPLES

### Example 1: Phenotype Potential Risk Model for Type I Diabetes

In this example, a target genome (FIG. 6, 51), consisting of an embryonic genome is input into the algorithm along with the genotype (FIG. 6, 31) and phenotype data (FIG. 6, 30) of each related parent (FIG. 6, 50). The genome and phenotype data consist of a binary variable E {0,1} representing the presence {1} or absence {0} of the Type-1 diabetes phenotype in each parent. Genomic properties (FIG. 6, 52) are calculated for each genome and consist of the presence {1} or absence {0} of a noted variant corresponding to each of seven genomic locations that affect Type-1 diabetes. This collection of 3×7=21 genomic properties (7 from each parent and 7 from the Target) are applied to the logistic model (FIG. 6, 44), described previously. This model accepts 23 parameters including the 21 genomic properties mentioned above and 2 related phenotype parameters, one from each related parent (FIG. 6, 30). Applying the 21 genomic parameters produces a distribution over the phenotype of probability of presence of target Type-1 Diabetes for each of the 4 combinations of related phenotypes ({0,1}×{0,1}) (FIG. 6, 53) as a potential phenotype risk. Next, the related parental phenotypes (FIG. 6, 30) are applied to the potential phenotype risk to resolve the two related phenotype parameters (FIG. 6, 54). A single phenotype risk distribution is produced (FIG. 6, 55), representing the probability of Type 1 diabetes for the studied embryo.

This process is repeated for many other health and other factors to create a risk profile. This profile, which is combined with the profiles resulting from analysis performed on a group of embryos, allows for the generation of a results panel which assists the decisions about which embryo(s) to implant during IVF.

### Example 2: Full Genome Risk Model for Type I Diabetes

In this example, the genomes are represented as full genome sequencing against human reference genome hg19 (GRCh37), and phenotype is a binary variable E {0,1} representing the presence of Type 1 diabetes in each organism. The training data is drawn from a collection of triplets each containing two parents and a biological child. The genomic properties are defined as 7 specified variants per genome, and the calculation of genomic properties represents the identification of the presence {1} or absence {0} of each of the 7 properties in each genome. While numerous methods of identifying phenotype potential risks may be used, a simple illustration involves augmenting the logistic regression method to include variables for the parental phenotype, producing a model logistic regression where parental phenotypes (4 parental phenotypes {0,1}×{0,1}) are individually alterable to produce differing phenotype risk profiles. This produces a potential phenotype risk that covers all of the 4 possible parental phenotypes. The actual parental phenotypes for each training example, are applied to the potential phenotype risk to generate a phenotype risk as a distribution over the possible phenotypes.

The distribution which is indicative of a probability of the phenotype occurring is compared with the actual target phenotype. The accuracy of the model in predicting actual target phenotypes is calculated and aggregated across all training samples and the model parameters adjusted by gradient descent until the stopping point is reached. The parameters for this logistic model then define the phenotype potential risk model.

This model is applied to additional embryos and allows for the generation of a results panel, which assists the decisions about which embryo(s) to implant during IVF.

### Example 3: Analysis of Embryonic Genome

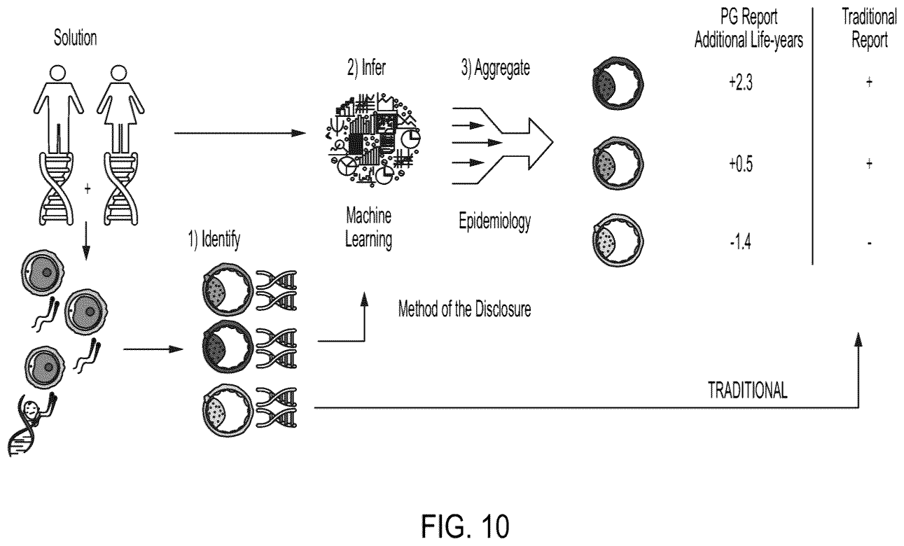

In this example, the entire genome of an embryo generated during IVF is analyzed using a three-step process prior to implantation (FIG. 10).

In the first step (FIG. 10, Step 1), embryonic genomic sequences are identified. This process begins by modeling the molecular processes by which each parent produces sperm and egg cells. Each normal adult cell contains two copies of each chromosome, one from the father (transmitted by sperm) and one from the mother (transmitted by the egg). However, the single copy of a chromosome provide by a parent is a mixture of the both of parental chromosomes, due to recombination.

Each chromosome contains approximately 50-300 million nucleotide pairs that can be arranged in a linear sequence. When a haploid (with only one chromosome copy) is produced, between zero and ten breaks (e.g., four breaks) are typically made. These breaks are sometimes referred to as “breakpoints”, or chiasmata in the biology literature, with each break swapping a contiguous segment of the source chromosome for a homologous segment from its homologous chromatid partner. The result is a single chromosome from a parent that is composed of a mosaic, or chimera, of their two homologous chromosomes.

As segments exchanged during crossover are typically rather large, often 10's of millions to 100's of millions of bases, a small amount of embryonic DNA can identify large stretches of parental DNA. As virtually all child DNA comes from one of the parents, such parental DNA sequencing can be used to fill-in missing sections from the smaller amount of embryonic DNA.

After the complete embryonic genome is assembled, the algorithms of the present disclosure, as described elsewhere, combine parental genome, environmental, and embryonic genome factors to identify risk probabilities inherited by the embryo across a wide spectrum of traits (FIG. 10, Step 2).

Once the risk probabilities of phenotypes are determined, the third step aggregates the distributions into a single score for each embryo. An example of such a score might be an expected change in quality adjusted life years for each embryo, aggregated across phenotypes, with each the probability of each phenotype contributing a component based on that phenotype's probability and the QALY impact of the phenotype. QALY impact of phenotypes may be derived using epidemiological impact data (FIG. 10, Step 3). A report is generated displaying the ranking of embryos ranked according to the score (FIG. 10, PG Report). In the above example the score represents the, expected health of a person who develops from the corresponding DNA in quality adjusted life-years (QALY). This single score gives a clear and interpretable measure of the expected health of a person maturing from this embryo. Additional risk weighting and phenotype impact details may also be presented in the report providing greater detail helpful for selecting one embryo from among the collection for implantation.

The embryo is ranked against other similarly-analyzed embryos. This aids parents undergoing IVF in making decisions between embryos with, for example, an embryo which exhibits a 10-fold increase in risk of Type I diabetes coupled with a normal (1-fold) risk of lung cancer versus an embryo which exhibits a normal (1-fold) risk of Type I diabetes coupled with a 10-fold increase in risk of lung cancer, and allows parents to choose embryos with the highest QALY score. This clarifies the parental selection process during IVF.

### Example 4: Comparison and Ranking of Two Embryos for Selection During IVF

In this example, disease risk distributions are combined with phenotype impact to identify a quantitative figure of merit (QALY). Disease risk traits provide a negative impact on the figure of merit while protective factors provide a positive impact on the QALY score. Two embryos, corresponding to two genomes, are compared and ranked resulting in the embryo with the higher figure of merit being selected for implantation during IVF.

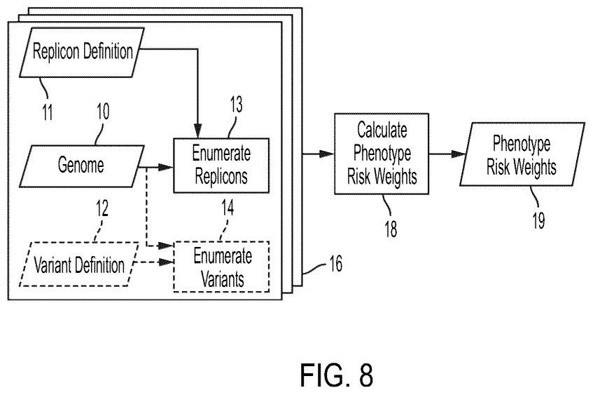

Software tools and Reference Data. Software tools which may be used are, e.g., Ubuntu Linux V20, bash shell; Samtools; bcftools; GLIMPSE imputation. Human reference genome hg19/GRCh37 is selected. The reference genome defines the shared coordinate system. Using a variety of human genomes, a genomic diversity reference panel is obtained. The genomic diversity reference panel is used to calculate the density of recombination events at breakpoints, which defines replicons (FIG. 8 and FIG. 11). Alternatively, a known genome reference panel, such as the one found at ftp.1000.genomes.ebi.ac.uk/vol1/ftp, can also be used. The reference genome contains phased information for a large number of individual genomes spanning a substantial fraction of human diversity. The reference panel is referred to as:
Reference.vcf

Step 1: Obtain Parental and Embryonic samples. To obtain parental and embryonic samples, a sperm provider (father) and egg provider (mother) visit a reproductive medicine clinic. A sperm sample is produced by the father and oocytes are retrieved from the mother for example, using the process described in Choe J, Archer J S, Shanks A L. In Vitro Fertilization. [Updated 2021 Sep. 9]. In: StatPearls [Internet]. Treasure Island (FL): StatPearls Publishing; 2021 January The sperm and eggs are processed to generate embryos which are subsequently processed for preimplantation genetic testing (PGT). Embryonic DNA samples are processed by isolating DNA molecules from the embryonic cells, preparing a DNA sequencing library, sequencing the library to produce embryonic reads, and computer processing the embryo-derived sequence reads. Samples are also taken from the father and mother for similar processing to obtain computer processed parental-derived sequence reads.

Step 2: Obtain aligned and called parental DNA information. Once the parental-derived sequence reads are amassed, complete parental variation information sequences are obtained (FIG. 10). Full parental genomic information allows for the identification of each nucleotide at every position in the reference genome coordinate system; the reference genome is used to identify genomic coordinates. This data can be provided in a text-based file format (e.g., VCF). The reference genome identifying genomic coordinates may be identified by examining the **reference record which might indicate a reference format such as “**reference=file: ///seq/references/hg19.fa.gz”, indicating the hg19 reference genome coordinate system. Parental genomes are referred to in the files:
Mother.vcf
Father.vcf

Step 3: Obtained aligned embryonic DNA. The embryonic-derived sequence reads are then aligned for each of N embryos. This data can be developed in the course of preimplantation genetic testing for aneuploidy (PGT-A) using whole genome sequencing (WGS) (FIG. 10, Step 1). Aligned data identifies the alignment reference (HG19) as well as the coordinates of each mapped read (a sequence of nucleic acids, generally 30-300 bases in length) in a sequence alignment and mapping (SAM) file, or its compressed, binary counterpart, a BAM file.

A dataset for embryonic DNA may contain 0.01 to 1.00-fold coverage of the genome consisting of 30 million to 3 billion individual nucleotides. This compares to the parental DNA information which may be distilled from 30 to 60-fold coverage (90 billion to 160 billion nucleotides).
Embryo_001.bam, Embryo_002.bam, . . . EmbryoN.bam

However, HG002.hs37d5.2×250.bam contains much more data than can typically be safely retrieved from a human embryo. To simulate characteristics of an embryonic sample it may be subsamples to simulate typical embryonic coverage such as 300 million nucleotides. This may be performed via the command:
samtools view-subsample

BAM files may be viewed using the samtools view command, in particular the alignment genomic reference be confirmed from the @PG header record displayed when providing the samtools option -H:
$ samtools view-H Embryo_001.bam
which will include a @ PG record showing the history commands that generated the .bam file, including the aligner and the reference genome, for example:

| | | | |
|---|---|---|---|
| @PG | ID:novoalign | PN:novoalign | VN:V3.02.07 |
| | CL:novoalign | | |

```
  -d /
  cluster/ifs/projects/Genomes/GIAB/refseqs/hs37d5.nd
x
  -f ../../fastq_2x250/D1_S1_L001_R1_001.fastq.gz
  ../../fastq_2x250/D1_S1_L001_R2_001.fastq.gz
  -F STDFQ --Q2Off -t 700 -o SAM -c 1
```

Here the text “hs37d5” indicates that the reference genome is hs37, which for this example may be interpreted as GRCh37 or equivalent to reference hg19.

Step 4: Impute Embryonic genome. The embryonic genome is generated from a combination of the information derived from the maternal and paternal DNA (FIG. 1B). This is done by augmenting the reference panel to contain the parental genomes:
$ bcftools merge -o Reference-aug.vcf \
   Reference.vcf Mother.vcf Father.vcf

The paternal and maternal derived sequence reads can then be used to determine the genomic sequence of the embryo. Variant calling, the process by which small nucleotide polymorphisms and other minor genomic changes are identified, is performed on the embryonic genome. As the embryonic genome has less genomic information than either the parental or reference genomes due to the limitations in sample gathering, error estimates at each genomic position in Embryo_001. vcf will be high. No embryonic read data may be available for many genomic positions.

```
$ bcftools mpileup -f hg19.fasta.gz -I -E -a ‘FORMAT/DP’
\
  -T sites.vcf Embryo_001.bam -Ou | \
  bcftools call -Aim -C alleles -T sites.tsv \
  -Oz -o Embryo_001.thin.vcf
```

To identify irregularities, the density of reads for each chromosome is used to identify aneuploidy, i.e., missing or excess chromosomes, and other genetic variations. The -r (region) flag with the samtools command:
samtools coverage Embryo_001.bam
   provides an estimate of read depth on each chromosome chromosomes with significantly fewer or more than normal number of are indications of aneuploidy and is used to test for the presence or an absence of an aneuploidy or a genetic variation of the embryo.

The next step is to perform imputation on the low-quality embryonic calls, conditioned on the parental data in the augmented reference panel. Source code, such as GLIMPSE, may be altered to ensure that the parents are included in each random sample, forcing the software to draw unknown genomic material from the parents, but crossover probabilities from the reference panel. Alternatively, it may be desirous to remove some of the non-parental genomes to focus inference on parental information. Alternatively, the code can be modified to force the inclusion of parental data at every random sampling of the reference:

$ GLIMPSE_phase --input Embryo_001.thin.vcf \
--reference Reference.vcf -- output
Embryo_001.full.vcf

This step specifically identifies breakpoints and breakpoint densities along the reference genome from the linkage disequilibrium in the reference panel. This step also comprises computer processing the embryo-derived sequence reads and the parental-derived sequence reads to determine the genomic sequence of the embryo.

Step 5: Computational analysis of embryonic genome to find phenotype risk profiles. Once the embryonic genome has been inferred, it is analyzed for key traits (FIG. 10, Step 2). Traits are inferred from the embryonic genome via genomic properties. The primary genomic property used in this example is the presence or absence of an alternate (non-reference) allele at each position in the inferred embryonic genome in accordance with (FIG. 1A, 80) an (FIG. 1A, 81). The collection of these genomic properties allows phenotype risk models to Calculate Phenotype Risks (FIG. 5, 41).

The embryonic genome is scanned for monogenic phenotypes, phenotypes determined by a single gene (e.g., sickle cell anemia, cystic fibrosis, Huntington disease, or Duchenne muscular dystrophy), by comparing the inferred genotype at each position to a list of known monogenic predictions. This identifies the probability of inherited monogenic traits from each parent, even when the trait is a disease. As the embryonic genome is fully phased, this also provides the number of alternate alleles when an alternate allele is present. Alternatively or additionally, the genome can be scanned for phenotype associated variants found in the SNPedia database to determine risk factors for variant phenotypes.

Once the embryonic sequence is identified (Embryo_001.full.vcf), the variants in this sequence may be used to identify phenotype trait risk factors, employing the PGS (Poly Genic Score) catalog (www.pgscatalog.org). Each trait is defined by an experimental factor ontology (EFO) ID.

An example model for EFO_0001360 corresponds to Type II diabetes mellitus:

# Citation = Vassy J L et al. Diabetes (2014).
doi: 10.2337/db13-1663

| rsID | chr_name | effect_allele | effect_weight | locus_name |
|---|---|---|---|---|
| rs12970134 | 18 | A | 0.0334 | MC4R |
| rs13233731 | 7 | G | 0.0043 | KLF14 |
| rs13389219 | 2 | C | 0.0374 | GRB14 |
| rs1801282 | 3 | C | 0.0453 | PPARG |
| rs2261181 | 12 | T | 0.0414 | HMGA2 |
| rs2943640 | 2 | C | 0.0414 | IRS1 |
| rs459193 | 5 | G | 0.0414 | ANKRD55 |
| rs780094 | 2 | C | 0.0334 | GCKR |
| rs8182584 | 19 | T | 0.0212 | PEPD |
| rs9936385 | 16 | C | 0.0531 | FTO |

The variants (effect_allele) possessed by the embryo are cumulated and produce a net log-odds ratio for the trait. The log-odds ratio is combined with the population risk for the trait (identified in the GHDx database to generate an absolute risk for this trait. This mapping may be performed by custom software or by a by services such as www.impute.me or selfdecode.com to provide risk estimates for each trait versus EFO code, based on the embryonic genome. Computer processing employs PGRS models trained with machine learning algorithms, particularly generalized linear models and logistic regression. Risk distributions are produced by applying the PRS models to embryonic genomes inferred from both parental genomic haplo-blocks.

Step 6. Combine phenotype risk profiles into a single figure of merit. The next step combines the numerous risk profiles generated for each embryonic genome during the previous step, into a single score suitable. Providing a single score (a PG report) for each embryonic genome allows the embryos to be ordered according to the score for preferred usage, including gestation or storage (FIG. 10, Step 3).

Several databases contain trait impacts in terms of epidemiological statistics, such as prevalence (people living with trait), incidents (new observations per year), DALY (disability adjusted life years lost) and YLL (years of life lost). These measure the burden or impact of having a trait, PRS measures the genomic risk for having a trait. By combining these values, we can estimate the expected impact of having a trait. PRS Trait risks are identified as probability of the reporting of an EFO trait.

The GHDx epidemiology global health database reports traits as Causes identified via ICD codes Jason L. Vassy et al, as “Polygenic Type 2 Diabetes Prediction at the Limit of Common Variant Detection”. The first step is to create a mapping between these terms identifying the GHDx ICD code or codes corresponding to each EFO trait. For example, “Type 2 Diabetes Mellites” is GHDx cause B.8.1.2 and corresponds to ICD10 code E11, which in turn maps to EFO 0001360, which has several PGRS models associated with it. This mapping can be found at github.com/EBISPOT/EFO-UKB-mappings.

DALYs are estimated on a population-wide scale and represent a deviation from a population-wide estimate of quality of life. However, populations are generally composed of three groups of people, people with a trait, people who will never have that trait and people who do not presently have the trait but will someday. While DALY measures the burden of disease on people who have it, it is also possible to estimate the value (anti-burden) of being resistant to a disease by considering the lifetime likelihood of getting a disease and the value of a reduction in the likelihood. Properly calibrated, PRS provide us both the probability of having a trait, and the probability of never having the trait. This allows for traits that are protective as well as traits that are dangerous.

If the total population is N and the people who will get a disease is N p and the per-capita DALY cost of having a disease is DALY p the total burden of having the disease is Nₚ×DALYₚ. However, the total quality of life is measured as a population average across both sick and well, estimating the perennially well N_{p̅} as N_{p̅}=N−Nₚ we have Nₚ×DALYₚ+N_{p̅}×DALY_{p̅}=D̅A̅L̅Y̅ₚ̅=0 therefore Nₚ×DALYₚ=N_{p̅}×DALY_{p̅} and

$${DALY}_{\overline{p}} = - \frac{{N}_{p} \times {DALY}_{p}}{N - {N}_{p}} .$$

Intuitively, if a few people get sick, there is a small advantage of being resistant to the illness. However, when a disease is widespread (e.g., Metabolic syndrome in the USA in 2020) the advantage of a protective factor is substantial.

Consider two diseases in a population. The first is diabetes with 5.3% of people getting it with an impact 6.96 DALY (quality adjusted life years lost). The impact being protected from it may be

$${DALY}_{\overline{p}} = - \frac{{N}_{p} \times {DALY}_{p}}{N - {N}_{p}} = - \frac{( N \times 5.3 \% ) \times 6.96}{N - N \times 5.3 \%} = - 0.389 ;$$

one may gain 0.398 quality adjusted life years if they knew they were protected from it. Now consider a second disease, cancer, with 1.1% of the population getting it, and an impact of 5.37 DALY. The impact being protected from it may be

$${DALY}_{\overline{p}} = - \frac{{N}_{p} \times {DALY}_{p}}{N - {N}_{p}} = - \frac{( N \times 1.1 \% ) \times 5.37}{N - N \times 1.1 \%} = - 0.0597 ;$$

one may gain 0.597 quality adjusted life years if they knew they were protected from it.

The net impact of all an embryo's genomic traits are tallied, and a PG report score is provided for each embryo. For example, the odds ratio produced by a PRS to an embryonic genome can produce the absolute probability of having a disease. When combined with disease frequency (Prevalence or Incidence, as appropriate) this can generate a change in expected probability of a trait t conditioned on genome G as P t (G), and the probability of not having the trait is P_{t̅}(G)=1−Pₜ(G) the expected impact of genomic on DALY is:

Pₜ(G)×DALYₜ+P_{t̅}(G)×DALY_{t̅}

For a given collection of traits T, with t E T the expected net impact is estimated as:

$$Score ( G ) = {\sum }_{t ∈ T} ( {P}_{t} ( G ) \times {DALY}_{t} + {P}_{\overline{t}} ( G ) \times {DALY}_{\overline{t}} )$$

Consider the same two diseases identified in step 6.2. Now assume there are two embryonic genomes:
G₁ with risk scores that offer a 9% chance of diabetes, but a 32% chance of cancer
G₂ with risk scores that offer a 22% chance of diabetes, but a 4% chance of cancer
These genomes can be scored using the formula:

Score(G₁)=[9%×6.96+(1−9%)×−0.389]+[32%×5.37+(1−32%)×−0.0597]=[0.6264+−0.354]+[1.7184+−0.0406]=1.950DALY

Score(G₂)=[22%×6.96+(1−22%)×−0.389]+[4%×5.37+(1−4%)×−0.0597]=[1.5312+−0.303]+[0.2148+−0.05731]=1.385DALY

Step 7. Rank embryos for further intervention based on score. Embryos can be ranked for further intervention based on score. For the two genomes described above:

Score(G₁)=1.950DALY=−1.950QALY,Score(G₂)=1.385DALY=−1.385QALY

Ranking these genomes according to QALY, Score(G₂)>Score(G₁) so it can be expected that the embryo corresponding to Genome 2 will have a healthier, longer life by nearly (−1.385 −−1.950)=0.565QALY or 12*0.565=6.8 QAL months.

This information is presents in the form of a PG report (FIG. 10), which is presented to an IVF patient. The patient, struggling to select between embryos, can chose the one with the higher QALY score, in this case, embryo 2, and have it implanted for procreation.

1.-119. (canceled)

120. A method for determining a genomic sequence of an embryo, comprising:
(a) isolating deoxyribonucleic acid (DNA) molecules from cells obtained or derived from a biopsy sample or culture sample of the embryo;
(b) preparing a sequencing library from the DNA molecules or derivatives thereof;
(c) sequencing the sequencing library to produce embryo-derived sequence reads; and
(d) computer processing the embryo-derived sequence reads to determine the genomic sequence of the embryo using sequence information derived from one or more parents of the embryo.

121. The method of claim 120, wherein the embryo is produced at least in part by in vitro fertilization of a sperm cell from a paternal subject and an egg cell from a maternal subject.

122. The method of claim 121, further comprising sequencing second DNA molecules obtained or derived from the paternal subject or the maternal subject to produce parental-derived sequence reads, wherein the parental-derived sequence reads comprise paternal-derived sequence reads from the paternal subject or maternal-derived sequence reads from the maternal subject, respectively, and wherein (d) further comprises computer processing the embryo-derived sequence reads and the parental-derived sequence reads to determine the genomic sequence of the embryo.

123. The method of claim 122, wherein (d) further comprises performing contig assembly of individual sequence reads of the embryo-derived sequence reads and the parental-derived sequence reads to determine the genomic sequence of the embryo.

124. The method of claim 123, wherein a portion of the genomic sequence of the embryo located between two breakpoints is determined based at least in part on a corresponding genomic sequence obtained from either the paternal-derived sequence reads or the maternal-derived sequence reads.

125. The method of claim 120, wherein the embryo is a human embryo.

126. The method of claim 120, wherein the embryo is a blastocyst.

127. The method of claim 126, wherein the biopsy sample comprises trophectoderm cells of the blastocyst.

128. The method of claim 120, wherein the culture sample comprises cells or cell-free DNA from culture media.

129. The method of claim 120, further comprising computer processing at least a portion of the genomic sequence of the embryo to determine a presence or an absence of an aneuploidy or a genetic variation of the embryo.

130. The method of claim 129, wherein the aneuploidy comprises trisomy 13, trisomy 18, trisomy 21, or a sex chromosome aneuploidy.

131. The method of claim 129, wherein the genetic variation comprises a monogenic variant associated with a variant phenotype.

132. The method of claim 131, wherein the variant phenotype comprises being affected by a disease or disorder or having an elevated risk of being affected by a disease or disorder.

133. The method of claim 131, further comprising determining a number of alleles of the embryo comprising the monogenic variant.

134. The method of claim 133, further comprising determining whether the embryo is affected or at elevated risk of being affected by the variant phenotype, unaffected or at reduced risk of being affected by the variant phenotype, or a carrier of the variant phenotype, based at least in part on the determined number of alleles of the embryo comprising the monogenic variant.

135. The method of claim 120, further comprising computer processing the genomic sequence of the embryo to determine a risk distribution of each of a set of phenotypes.

136. The method of claim 135, wherein computer processing the genomic sequence of the embryo comprises using a trained machine learning algorithm.

137. The method of claim 136, wherein the trained machine learning algorithm comprises a neural network, a support vector machine, a random forest, a generalized linear model, or a logistic regression.

138. The method of claim 135, wherein the risk distribution for a phenotype of the set of phenotypes is determined based at least in part on a combination of at least one of paternal haplo-blocks inherited by the embryo, maternal haplo-blocks inherited by the embryo, an observable paternal phenotype, and an observable maternal phenotype.

139. The method of claim 135, further comprising computer processing the risk distributions of the set of phenotypes into a quantitative figure of merit indicative of an expected health of an offspring that develops from the embryo.

140. The method of claim 139, wherein each of the risk distributions of the set of phenotypes contributes a positive expected value, a negative expected value, or a zero expected value toward the quantitative figure of merit.

141. The method of claim 139, wherein the quantitative figure of merit comprises an expected number of quality adjusted life years of the offspring.

142. The method of claim 139, further comprising determining a quantitative figure of merit for each of a plurality of embryos.

143. The method of claim 142, wherein the quantitative figures of merit for the plurality of embryos are determined using a user-selected set of weights for each of at least one of the set of phenotypes.

144. The method of claim 142, further comprising selecting, ordering, or ranking individual embryos of the plurality of embryos based at least in part on the quantitative figures of merit for the individual embryos.

145. The method of claim 144, further comprising implanting the selected embryo into the female subject, based at least in part on the selecting, the ordering, or the ranking.

146. The method of claim 120, wherein the sequencing library in (b) is prepared without use of nucleic acid amplification.

147. The method of claim 120, wherein the genomic sequence of the embryo is at least 90% of a whole genomic sequence of the embryo.

148. A method for providing a selection of an embryo from a set of sibling embryos, comprising:
(a) obtaining a first sequence data set generated upon sequencing one or more nucleic acid molecules obtained from the embryo, which first sequence data set is not a whole genome of said embryo;
(b) computer processing the first sequence data set with sequence information obtained from one or more parents of the sibling embryos to yield a second sequence data set, which second sequence data set spans a greater genomic window than the first sequence data set; and
(c) computer processing the second sequence data set or derivative thereof to provide the selection of said embryo from the set of sibling embryos.

149. A non-transitory computer-readable medium comprising machine-executable code that, upon execution by one or more computer processors, implements a method for determining a genomic sequence of an embryo, the method comprising:
(a) receiving embryo-derived sequence reads of an embryo, wherein the embryo-derived sequence reads are generated by sequencing deoxyribonucleic acid (DNA) molecules that are isolated or derived from cells obtained or derived from a biopsy sample or a culture sample of the embryo;
(b) receiving sequence information derived from one or more parents of the embryo; and
(c) processing the embryo-derived sequence reads to determine the genomic sequence of the embryo using the sequence information derived from the one or more parents of the embryo.
